(19) United States
(12) Patent Application Publication
(10) Pub. No.: US 2022/0107329 A1
(43) Pub. Date: Apr. 7, 2022

(54) METHODS OF DETECTING AND DIAGNOSING DEFECTS OF THE LYMPHATIC VASCULATURE

(71) Applicants: NORTHWESTERN UNIVERSITY, Evanston, IL (US); THE BOARD OF TRUSTEES OF THE LELAND STANFORD JUNIOR UNIVERSITY, Stanford, CA (US)

(72) Inventors: Guillermo Oliver, Chicago, IL (US); Stanley Rockson, Stanford, CA (US)

(21) Appl. No.: 17/489,467

(22) Filed: Sep. 29, 2021

Related U.S. Application Data
(60) Provisional application No. 63/086,448, filed on Oct. 1, 2020.

(51) Int. Cl. G01N 33/86

(52) U.S. Cl. CPC G01N 33/86; G01N 2333/521

(57) ABSTRACT

Disclosed herein are methods and compositions useful for identifying and treating diseases, conditions, and abnormalities of the lymphatic vasculature. In some embodiments, the methods include determining a level of platelet factor 4 (PF4) in a biological sample of the subject, and diagnosing the subject as having a disease or condition of the lymphatic vasculature if the subject's PF4 level is increased compared to a control PF4 level. Also disclosed herein are method of treating a subject having an increased PF4 level. In some embodiments, the methods include administering a composition comprising a molecule that specifically binds to and inhibits the function of PF4, such as, for example, an antibody. In some embodiments, the subject has previously been diagnosed with obesity.

### CROSS-REFERENCING

This application claims the benefit of U.S. provisional application Ser. No. 63/086,448, filed on Oct. 1, 2020, which application is incorporated by reference by reference.

### GOVERNMENT RIGHTS

This invention was made with Government support under contracts RO1HL073402 and T32 HL134633 awarded by the National Institutes of Health. The Government has certain rights in the invention.

### BACKGROUND

The lymphatic vasculature is a network of thin-walled initial lymphatic capillaries and larger collecting vessels covered by a continuous layer of endothelial cells providing a unidirectional conduit for filtered tissue interstitial fluids, metabolites, macromolecules and cells toward the central venous circulation. Its principal function is to maintain fluid homeostasis by removing the protein-enriched fluids from the extracellular space and returning them, in the form of lymph, to the bloodstream. Lymphatics are also important for lipid transport and immune cell trafficking, among other functions.

One of the main disorders that ensue from malfunction of the lymphatic vasculature is lymphedema, a disfiguring, disabling, and occasionally, life-threatening clinical condition characterized by the localized interstitial accumulation of protein-rich fluid, thereby promoting tissue edema for which, at present, treatment options are few and efficacy is limited. This disease affects millions of persons worldwide, and most commonly entails swelling of the extremities, tissue fibrosis, susceptibility to infections and accumulation of subcutaneous fat. Lymphedema can result from either primary or acquired (secondary) disorders. Primary lymphedema is the consequence of genetic defects that impact the formation and normal function of the lymphatic vasculature, and most commonly manifests during infancy, childhood or adolescence. Secondary lymphedema is the more common presentation and is caused by lymphatic trauma sustained after surgery, radiation therapy, infection or trauma. In general, overt lymphedema can be diagnosed based on the clinical context and the physical examination; however, more precise staging and characterization requires imaging protocols that are often invasive.

A direct correlation and mechanistic relationship between the lymphatic vasculature and the adipose compartment have recently been recognized in patients with lymphatic disorders. Abnormal subcutaneous fat accumulation in the affected edematous regions in patients with secondary lymphedema is the inescapable consequence of sustained defective lymphatic drainage. Analysis of patients has also shown that malformation of cutaneous lymphatics causes bilateral fat accumulation in the thigh and buttock, a phenotype that worsens during puberty, while dermal lipid accumulation occurs in idiopathic lymphedema patients. Although physiotherapy and use of compression garments do limit interstitial fluid accumulation, at present there are limited options for the treatment of these more advanced manifestations of the disease.

Lipedema is a common, chronic lymphovascular disease characterized by bilateral, symmetrical swelling in the extremities due to the deposition of abnormal subcutaneous adipose tissue. Lipedema, often misdiagnosed as obesity or lymphedema, occurs almost exclusively in females and has a likely genetic component, as a positive family history is common. Nevertheless, in contrast to lymphedema, overt interstitial edema is not observed in lipedema, and the swelling due to adipose hypertrophy occurs in a distinctly symmetrical pattern. Early studies by Bilancini et al., demonstrated that lipedema is consistently associated with functional alterations of the lymphatic vasculature. Using dynamic imaging, they showed that patients suffering from lipedema have an abnormal lymphoscintigraphic pattern, with a slowing of the lymphatic flow similar to the alterations found in lymphedema patients. Despite these insights, lipedema is frequently misdiagnosed as obesity or lymphedema, and the pathogenesis and molecular mechanisms of this disease are still very poorly understood. Nevertheless, lipedema appears to be an adipose disorder with an apparent contribution of lymphatic malfunction. Whether those lymphatic alterations are partially responsible for the disease, or are secondary to the related obesity features is not yet known. Unfortunately, even with focused morphological analysis, lipedema is not easy to differentiate from obesity; clinicians often lack familiarity with this condition, distinct clinical imaging attributes have not been identified, there are no known biomarkers for the disease, and conclusive mechanistic evidence supporting the proposal that lymphatic defects contribute to the disease is still lacking.

More recently, the functional roles of the lymphatic vasculature have broadened. New evidences suggest that asymptomatic defective and/or leaky lymphatic vessels could be responsible for certain forms of obesity, inflammatory bowel disease/Crohn's disease, glaucoma and some forms of neurological pathology. Thus, identification of easily accessible, reliable biomarkers of lymphatic malfunction would be a valuable resource to assist not only in the conclusive diagnosis of lymphedema, but also to facilitate the differential diagnosis among lymphedema, lipedema and obesity subjects. Furthermore, the identification of such biomarkers could eventually also help to identify and diagnose subtle, asymptomatic lymphatic alterations that might contribute to some of the aforementioned disorders. Accordingly, we profiled and compared circulating exosomes isolated from blood plasma from animal models and from patients with and without documented lymphatic pathologies. Exosomes are small vesicles (30-100 nm in diameter) of endocytic origin secreted by most cells (including endothelial cells). These extracellular vesicles contain cell type-specific proteins and genetic materials, including mRNAs, miRNAs and DNA. They can also exert a functional influence once taken up by recipient cells, therefore representing novel mediators of intercellular communication. Exosomes are emerging biomarkers of various types of diseases.

Genetic or acquired defects of the lymphatic vasculature often result in disfiguring, disabling and, occasionally, life-threatening clinical consequences. Diagnostic tools for all of the lymphatic disorders are relatively limited, relying chiefly on rather cumbersome and expensive imaging techniques. There has been a historical desire to identify biomarkers that might assist in the diagnostic approach to patients with chronic edema (estimated 5-10 million in the U.S.). In addition, lipedema, a cryptogenic disease with lymphatic features, is estimated to affect 11% of the U.S. female population. For these patients, there are no tools for objective validation of differential diagnosis. All of the lymphatic entities are frequently misdiagnosed as obesity, leading to delays in diagnosis and treatment and, conversely, the obese population in the U.S. likely harbors a substantial cohort of patients whose obesity exists on the basis of lymphatic pathology. When imaging is utilized, it is cumbersome, often painful, and very expensive. This is particularly true for the lymphatic vascular defects that otherwise lack readily utilized diagnostic features. Accordingly, there is a need in the art for methods to accurately identify and diagnose subjects suffering from abnormalities of the lymphatic vasculature.

### SUMMARY

Disclosed herein are methods and compositions useful for identifying and treating diseases, conditions, and abnormalities of the lymphatic vasculature. In some embodiments, the methods include determining a level of platelet factor 4 (PF4) in a biological sample of the subject, and diagnosing the subject as having a disease or condition of the lymphatic vasculature if the subject's PF4 level is increased compared to a control PF4 level. Also disclosed herein are method of treating a subject having an increased PF4 level. In some embodiments, the methods include administering a composition comprising a molecule that specifically binds to and inhibits the function of PF4, such as, for example, an antibody. In some embodiments, the subject has previously been diagnosed with lymphedema, lipedema, obesity, has suffered a lymphatic trauma, has undergone a surgery, or has undergone a cancer treatment such as radiation therapy or lymph node removal.

### BRIEF DESCRIPTION OF THE DRAWINGS

FIG. 1. Characterization of plasma exosomes from young and old Prox1^{+/-} mice. Exosome particle concentration is compared between young and old WT and Prox1^{+/-} mice (N=4-6). Data represent mean value ±standard error of the mean (s.e.m) and statistical analyses performed by unpaired t test. *P<0.05, ****P<0.0001.

FIG. 2. Protein signatures in plasma exosomes from young and old Prox1^{+/-} mice. Proteins that are both increased (A) or decreased (B) in young and old Prox1^{+/-} mice were compared to age-matched WT mice. Gene name in red highlights the common changes in Ob/Ob mice. (N=4-6)

FIG. 3. Proteomic analysis of plasma exosomes from Ob/Ob mice compared to WT controls. (A) Pie chart shows up and downregulated protein changes in Ob/Ob mice compared to WT controls. (N=3) (B-C) Venn diagram shows the common and unique proteins in Prox1^{+/-} compared to Ob/Ob mice. The common proteins are presented in red fonts in FIG. 2 A-B.

FIG. 4. Validation of PF4 levels in plasma exosomes from individuals with normal lymphatics and patients with lymphatic disorders. (A) ELISA quantification of PF4 levels in exosomes from control subjects and indicated groups of patients. PF4 levels were normalized to the exosome protein content. (red dots indicate outliers detected by Iterative Grubb's test and are excluded from the statistical analysis. * indicates P≤0.05, ** indicates P≤0.01, *** indicates P≤0.001 compared to control.) (B) ROC curve of PF4 for each diagnosis. The cutoff value of PF4 with sensitivity and specificity, as well as AUC and CI are presented in the separate table below the figure. (AUC, area under the receiver operating characteristic curve; CI, confidence interval.) (C) The PF4 from individuals with normal lymphatics are further divided based on BMI >30, and the PF4 level from lean and obese individuals with normal lymphatics is not statistically different (red dot indicates the outlier in FIG. 4A normal group detected by Iterative Grubb's test group and is excluded from the statistical analysis). (D) The PF4 from individuals with secondary lymphedema, lymphovascular disease and lipedema are grouped into lean and obese based on BMI of 30, and the PF4 level from lean and obese individuals with lymphatic disorders is not statistically different (red dot indicates the outlier in FIG. 4A lymphovascular disease group detected by Iterative Grubb's test group and are excluded from the statistical analysis).

### DETAILED DESCRIPTION

The present invention is described herein using several definitions, as set forth below and throughout the application.

As used in this specification and the claims, the singular forms “a,” “an,” and “the” include plural forms unless the context clearly dictates otherwise. For example, the term “a polypeptide fragment” should be interpreted to mean “one or more a polypeptide fragment” unless the context clearly dictates otherwise. As used herein, the term “plurality” means “two or more.”

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

As used herein a “subject sample” or a “biological sample” from the subject refers to a sample taken from the subject, such as, but not limited to a tissue sample (e.g, fat, muscle, skin, tumor, etc.) or fluid sample (e.g., saliva, blood, serum, plasma), and or cells or sub-cellular structures such as vesicles and exosomes. In some embodiments, a biological sample comprises blood, blood plasma, or blood plasma exosomes.

The disclosed methods and compositions may be utilized to treat a subject in need thereof. A “subject in need thereof” is intended to include a subject having or at risk for developing diseases or disorders of the lymphatic vasculature. In some embodiments, a subject in need thereof is likely to suffer, is suffering, or has suffered from one or more of the following symptoms: swelling of part of an arm, leg, breast, or torso; a feeling of heaviness or tightness in part of an arm, leg, breast, or torso; restricted range of motion in an arm or leg; fibrosis; numbness or tingling in an arm or leg; disproportionate fat below the waist, especially in the thigh and buttock; bilateral symmetrical swelling in the extremities due to the deposition of abnormal subcutaneous adipose tissue; accumulation of subcutaneous fat, especially in edematous regions; susceptibility to infections. In some embodiments, a subject in need thereof has suffered a lymphatic trauma, e.g., an injury, a surgery, or a cancer treatment, e.g., a lymph node removal or radiation therapy. In some embodiments, a subject in need thereof is genetically predisposed to developing diseases or disorders of the lymphatic vasculature. In some embodiments, a subject in need thereof is diagnosed with a disease or condition of the lymphatic vasculature such as, but not limited to lymphedema, lipedema, obesity, inflammatory bowel disease/Crohn's disease, glaucoma, some forms of neural pathology, cellulitis, cancer, Hodgkin's Disease/Hodgkin's Lymphoma. Non-Hodgkin's Lymphoma, lymphadenitis, lymphangitis. Lymphocytosis. Additional examples of intrinsic lymphatic diseases, include but are not limited to lymphatic vascular malformation, chylous effusions, central conducting lymphatic anomaly, Kaposiform lymphangiomatosis, protein-losing enteropathy. For general pathology that may include a lymphatic component, examples include, but are not limited to atherosclerosis, congestive heart failure, and diabetes mellitus

### Polynucleotides

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

In some embodiments, therapeutic nucleic acids are employed, e.g., to decrease the level of circulating PF4 in a subject in need thereof. In some embodiments, the therapeutic nucleic acid includes one or more of an antisense oligonucleotide; DNA aptamers; gene therapy; micro RNAs; short interfering RNAs; ribozymes; RNA decoys; and circular RNAs. Two transcript (mRNA) sequences of PF4 are provided as SEQ ID NO: 3 and SEQ ID NO: 4. Therapeutic nucleic acids can be made by methods well known in the art.

### Polypeptides

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein.

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. A “variant” of a reference polypeptide sequence may include a conservative or non-conservative amino acid substitution relative to the reference polypeptide sequence,

The disclosed peptides may include an N-terminal esterification (e.g., a phosphoester modification) or a pegylation modification, for example, to enhance plasma stability (e.g. resistance to exopeptidases) and/or to reduce immunogenicity.

A “deletion” refers to a change in a reference amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence (e.g., a fusion partner) that extends the half-life of the fusion polypeptide in the tissue of interest, such as serum, plasma, fatty tissue, lymph. A “variant” of a reference polypeptide sequence may include a fusion polypeptide comprising the reference polypeptide.

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

### Platelet Factor 4

The disclosed methods of diagnosis and treatment utilize and/or include a Platelet Factor 4 (PF4) polypeptide or a functional fragment or variant thereof, or a nucleotide sequence encoding a PF4 polypeptide or a functional fragment or variant thereof.

The amino acid sequence of human PF4 isoform 1 is provided as SEQ ID NO: 1. The amino acid sequence of human PF4 isoform 2 provided as SEQ ID NO: 2. Human HP4 transcript variant 1 is provided as SEQ ID NO: 3, and Human HP4 transcript variant 2 is provided as SEQ ID NO: 4.

SEQ ID NO: 1

| | |
|---|---|
| 1 | mssaagfcas rpgllflgll llplvvafas aeaeedgdlq |
| | clcvkttsqv rprhitslev |
| 61 | ikagphcpta qliatlkngr kicldlqapl ykkiikklle s |

SEQ ID NO: 2

| | |
|---|---|
| 1 | mitatlngep aeclatvpga apapptwleq llsgggviya |
| | eaeedgdlqc lcvkttsqvr |
| 61 | prhitslevi kagphcptaq liatlkngrk icldlqaply |
| | kkiikklles |

SEQ ID NO: 3

| | |
|---|---|
| 1 | attggccaca gagacccagc ccgagtttcc catcgcactg |
| | agcactgaga tcctgctgga |
| 61 | agctctgccg cagcatgagc tccgcagccg ggttctgcgc |
| | ctcacgcccc gggctgctgt |
| 121 | tcctggggtt gctgctcctg ccacttgtgg tcgccttcgc |
| | cagcgctgaa gctgaagaag |
| 181 | atggggacct gcagtgcctg tgtgtgaaga ccacctccca |
| | ggtccgtccc aggcacatca |
| 241 | ccagcctgga ggtgatcaag gccggacccc actgccccac |
| | tgcccaactg atagccacgc |
| 301 | tgaagaatgg aaggaaaatt tgcttggacc tgcaagcccc |
| | gctgtacaag aaaataatta |
| 361 | agaaactttt ggagagttag ctactagctg cctacgtgtg |
| | tgcatttgct atatagcata |
| 421 | cttctttttt ccagtttcaa tctaactgtg aaagaacttc |
| | tgatatttgt gttatcctta |
| 481 | tgattttaaa taaacaaaat aaatcaagtt gtagtatagt |
| | caaaatactt cttaataata |
| 541 | gtgcaaaaat tgtgttgaca cataacaatt tcatggaaga |
| | aaaaaattcc ggtattttaa |
| 601 | gcaaaaagta ttttgaagga aggtgtgaat actggttatg |
| | cttggtgtta catgttggct |
| 661 | gatacatatt catgcattta catgattgca gtactttata |
| | gctacatatt taccttgacc |
| 721 | attattatta cctttgccaa taaatatcag taacacagat |
| | ggcttttaaa aaa |

SEQ ID NO: 4

| | |
|---|---|
| 1 | atcttagttt ccgcaccgca gttcctcggt gtccacttca |
| | ggcttccgga ctggaaggac |
| 61 | agccgggaat aaaacgtgcc ggcgaggctc aggagtcatt |
| | ggccacagag acccagcccg |
| 121 | agtttcccat cgcactgagc actgagatcc tgctggaagc |
| | tctgccgcag catgagctcc |
| 181 | gcagccgggt tctgcgcctc acgccccggg ctgctgttcc |
| | tggggttgct gctcctgcca |
| 241 | cttgtggtcg ccttcgccag cggtgagagc agaagccagg |
| | ctgtgagggc tggcagcggc |
| 301 | gagggggagt ccgggaagcc ctggggctgg ggaggaatcc |
| | tctaggatca tgatcacagc |
| 361 | cacacttaac ggagagcctg ctgagtgtct ggccacagtg |
| | ccaggcgctg cacctgcacc |
| 421 | tcccacctgg ttagaacaac ttctgtctgg gggaggtgtg |
| | atttatgctg aagctgaaga |
| 481 | agatggggac ctgcagtgcc tgtgtgtgaa gaccacctcc |
| | caggtccgtc ccaggcacat |
| 541 | caccagcctg gaggtgatca aggccggacc ccactgcccc |
| | actgcccaac tgatagccac |
| 601 | gctgaagaat ggaaggaaaa tttgcttgga cctgcaagcc |
| | ccgctgtaca agaaaataat |
| 661 | taagaaactt ttggagagtt agctactagc tgcctacgtg |
| | tgtgcatttg ctatatagca |
| 721 | tacttctttt ttccagtttc aatctaactg tgaaagaact |
| | tctgatattt gtgttatcct |
| 781 | tatgatttta aataaacaaa ataaatcaag ttgtagtata |
| | gtcaaaatac ttcttaataa |
| 841 | tagtgcaaaa attgtgttga cacataacaa tttcatggaa |
| | gaaaaaaatt ccggtatttt |
| 901 | aagcaaaaag tattttgaag gaaggtgtga atactggtta |
| | tgcttggtgt tacatgttgg |
| 961 | ctgatacata ttcatgcatt tacatgattg cagtacttta |
| | tagctacata tttaccttga |
| 1021 | ccattattat tacctttgcc aataaatatc agtaacacag a |

PF4 is a small cytokine belonging to the CXC chemokine family that is also known as a chemokine (C-X-C motif) ligand 4 (CXCL4). This chemokine is released from alpha granules of activated platelets during platelet aggregation, and promotes blood coagulation by moderating the effects of heparin-like molecules. Due to these roles, it, is predicted to play a role in wound repair and inflammation. It is usually found in a complex with proteoglycan. The heparin:PF4 complex is the antigen in heparin-induced thrombocytopenia, an idiosyncratic autoimmune reaction to the administration of the anticoagulant heparin. PF4 autoantibodies have also been found in patients with thrombosis and features resembling HIT but no prior administration of heparin.

### Identifying Disease and Conditions of the Lymphatic Vasculature by Determining PF4 Levels

Measuring circulating levels of PF4 has broad applicability in the clinical assessment of lymphedema, lymphovascular disease, and lipedema, where objective validation of differential diagnosis is scanty or completely lacking. In addition, this biomarker for lymphatic disease may be utilized to identify asymptomatic lymphatic malfunction responsible for a broad array of human pathology, including obesity, endocrine, neurological, and cardiac disorders, and inflammatory bowel disease, among others.

The blood plasma exosomes isolated from mouse models and from human subjects have been profiled and compared among samples obtained from subjects with and without symptomatic lymphatic pathologies. Platelet factor 4 (PF4/CXCL4) was identified as a biomarker that reliably identifies lymphatic dysfunction in all categories, including lipedema, and discriminates these disorders from obesity. As a circulating protein, assay of PF4, when scaled for clinical use, will provide an invaluable diagnostic platform for common and uncommon lymphatic diseases, along with an array of human disease in which lymphatic involvement is now recognized.

The present technology has immediate and applicable advantages over current diagnostic methods. Diagnostic tools for all of the lymphatic disorders are relatively limited or non-existent. Current diagnosis relies chiefly on rather cumbersome and expensive imaging techniques, if they are available for the clinical entity in question. When imaging is utilized, it is cumbersome, often painful, and very expensive. This is particularly true for the lymphatic vascular defects that otherwise lack readily utilized diagnostic features. The possibility of using a reliable biomarker present in patients' serum will facilitate the diagnoses of lymphedema, lipedema, lymphatic and complex vascular malformations, and other lymphovascular diseases, along with associated co-morbidities

The present technology provides a real-world solution to a problem facing many patents, and fulfills and unmet need. Lymphatic disease is common and highly morbid, but it is often unrecognized, misdiagnosed, or recognized at a relatively advanced stage, when treatment options are more limited. One explanation for this problem is the lack of readily accessible, accurate and relatively non-invasive diagnostic technologies. For lipedema, a very common disorder among adult women, there are no diagnostic tools at all. This invention provides a readily commercialized clinical assay that will accurately discriminate the presence of these disorders. It has broad applicability for implementation and represents a large potential market.

Thus, by determining a subject's PF4 level and comparing it to a control PF4 level, a determination can be made regarding the subject's lymphatic vasculature, e.g., whether there is damage, malformation, and/or aberrant function. This information can then be used, combined with clinical data (such as sex, age, height, weight, prior medical history, prior and current treatments or therapies, molecular/genetic data, edema status and location, subcutaneous fat accumulation, tissue fibrosis, etc.) to assist physicians in making a more accurate diagnosis, and to direct treatment.

A subject's PF4 levels can be determined by obtaining a biological sample from the subject, and then testing for PF4 level (e.g., PF4 mRNA or protein) in the sample. In some embodiments, the sample comprises a blood sample, a blood plasma sample, or a blood plasma exosome sample.

Methods to determine PF4 protein levels include, but are not limited to: immunoassays assays, such as ELISA and Western blotting; chromatographic methods; and protein mass spectrometry assays. Antibodies that bind to PF4 are well-known in the art and are commercially available, as are PF4 ELISA kits. By way of example, but not by way of limitation, as disclosed herein, total protein quantification in exosomes was performed using the BCA protein assay kit (Pierce, Thermo Fisher Scientific). PF4 concentration in exosomes was quantified using a human PF4 ELISA kit (R&D Systems, Bio-Techne).

Extracellular vesicles, found in all biofluids, include exosomes (30 nm to 150 nm) from endosomes/multivesicular bodies and microvesicles (150 nm to 1000 nm) from the plasma membrane. Various methods for the isolation of exosomes from biological fluids have been developed. They include centrifugation, chromatography, filtration, polymer-based precipitation and immunological separation. See, e.g., Patel et al (Scientific Reports 2019 9: 5335).

Additionally or alternatively, PF4 levels can be determined by evaluating a subject's PF4 RNA (mRNA) levels. Methods to detect RNA are well known in the art, and numerous kits and options are commercially available. By way of example, but not by way of limitation, methods include reverse transcription and polymerase chain reaction, (RT-PCR), methods employing direct oligonucleotide probe hybridization to PF4 RNA e.g., Northern blotting.

As used herein, the term control sample, control level, or control subject, refer to a sample, level, or subject that is considered “normal” or “wild-type” relative to the specific condition or conditions under investigation. For example, a PF4 control level is the level of PF4 identified in a subject or a cohort of subjects (e.g., pooled samples, or averaged values) that are not symptomatic for, and have no known precondition for developing a disease or condition of the lymphatic vasculature (e.g., a “healthy” subject, and/or a subject without lymphatic pathology). In some embodiments, a control PF4 level is characterized as about 1-10 ng/μ1 of exosome total protein.

As described herein, an elevated circulating PF4 level (e.g., the PF4 level in plasma exosomes) is indicative of a disruption of the lymphatic vasculature. In some embodiments, an elevated PF4 level is characterized as about 2 fold greater than a control PF4 level; about 3 fold, about 4 fold, or about 5 fold greater than a control PF4 level.

In any embodiment, the level of PF4 may be normalized to a control marker, e.g., a constitutive protein or RNA.

In some embodiments, a subject's PF4 levels can be determined before, during, and/or after a course of treatment or therapy, or throughout the subject's life, e.g., if a genetic predisposition exists or if clinical symptoms appear.

In some embodiments, after a subject has been identified as having elevated PF4 levels, the subject may be subject to further diagnostic methods and/or treatment methods for diseases and conditions of the lymphatic vasculature. Further diagnostic methods may include, but are not limited to an magnetic resonance imaging scan of the subject's lymphatic vasculature system to identify obstructions; a computer tomography scan of the subject's lymphatic vasculature system to identify obstructions; a Doppler ultrasound procedure to identify obstructions in the subject's lymphatic vasculature system; and/or radionuclide imaging of the subject's lymphatic system (lymphoscintigraphy) to identify obstructions. Treatment methods may include, but are not limited to: exercise, such as light exercises in which the subject moves their affected limb to encourage lymph fluid drainage; wrapping the subject's affected limb with bandages to encourage lymph fluid to flow back toward the trunk; massage to promote lymph drainage; pneumatic compression to promote lymph drainage; wearing compression garments; and complete decongestive therapy (CDT).

In measuring the biomarker, the absolute amount of the biomarker may be determined, or the amount of each biomarker relative to one or more control markers (which may be constitutive, for example) may be determined. Whether the amount of a biomarker is increased or decreased may be in relation to the amount of the biomarker (e.g., the average amount of the transcript) in control samples (e.g., in blood samples collected from a population of at least 100, at least 200, or at least 500 subjects that are known or not known to have the disease or condition.

In some embodiments, the method may comprise providing a report indicating whether the subject has the disease or condition based on the measurements of the amount of the biomarker, optionally with the amounts of other biomarkers and other symptoms. In some embodiments, this step may involve calculating a score based on the amount of the biomarer, where the score correlates with the phenotype and can be a number such as a probability, likelihood or score out of 10, for example. In these embodiments, the method may comprise inputting the amount of the biomarker into one or more algorithms, executing the algorithms, and receiving a score for each phenotype based on the calculations. In these embodiments, other measurements from the subject, e.g., whether the subject is male, the age of the subject, etc. may be input into the algorithm.

In some embodiments, the method may involve creating the report e.g., in an electronic form, and forwarding the report to a doctor or other medical professional to help identify a suitable course of action, e.g., to identify a suitable therapy for the subject. The report may be used along with other metrics as a diagnostic to determine whether the subject has the disease or condition.

In any embodiment, report can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infrared transmission channels as well as a network connection to another computer or networked device, and the internet or including email transmissions and information recorded on websites and the like. In certain embodiments, the report may be analyzed by an MD or other qualified medical professional, and a report based on the results of the analysis of the image may be forwarded to the subject from which the sample was obtained.

### Kits

As disclosed herein, blood plasma exosomes isolated from mouse models and from human subjects have been profiled and compared with and without symptomatic lymphatic pathologies. Platelet factor 4 (PF4/CXCL4) was identified as a biomarker that reliably identifies lymphatic dysfunction in all categories, including lipedema, and discriminates these disorders from obesity. Accordingly, kits can be designed and used to identify lymphatic defects by elevated levels of PF4. Such kits can be designed to detect PF4 levels in blood, blood plasma, or in blood plasma exosomes. Moreover, either PF4 protein or PF4 RNA levels may be detected and quantified or determined. In some embodiments, both PF4 protein, and PF4 RNA levels are determined.

Development of a commercially viable assay (kits) for PF4 will have immediate applicability in this large patient population. In addition, recent evidence suggests that otherwise asymptomatic defective lymphatic vasculature likely contributes to an array of other pathologies, including obesity, inflammatory bowel disease and neurological disorders, among others. Accordingly, identification of biomarkers of lymphatic malfunction will provide a valuable resource for the diagnosis and clinical discrimination of lymphedema, lipedema, obesity and other potential lymphatic-related pathologies.

Accordingly, in some embodiments, kits for the detection of PF4 levels are provided, wherein the kit includes one or more of a PF4 antibody to detect a PF4 protein level, or may include other detection means to detect a PF4 RNA expression level. Such kits may include components for PF4 RNA amplification, PF4 RNA reverse transcription, and/or PF4 RNA or cDNA hybridization. Kits may also include and control samples and instructions for use.

### Diseases and Conditions of the Lymphatic Vasculature

Provided herein are compositions and methods useful to identify and treat conditions, disease or injuries of the lymphatic vasculature. Subjects suitable for the disclosed methods of diagnosis and treatment may include, but are not limited to, subjects having or at risk for developing disease, conditions or injury that negatively affects the lymphatic vasculature. In some embodiments, subjects may be asymptomatic for a lymphatic disorder, yet such disorder may be exacerbating a co-existing disease or condition. By way of example, but not by way of limitation, in some embodiments, subjects may be suffering from, or at risk of swelling of part of an arm, leg, breast, or torso; a feeling of heaviness or tightness in part of an arm, leg, breast, or torso; restricted range of motion in an arm or leg; fibrosis; numbness or tingling in an arm or leg; disproportionate fat below the waist, especially in the thigh and buttock; bilateral symmetrical swelling in the extremities due to the deposition of abnormal subcutaneous adipose tissue; accumulation of subcutaneous fat, especially in edematous regions; susceptibility to infections, obesity. In some embodiments, a subject has suffered a lymphatic trauma, e.g., an injury, a surgery, or a cancer treatment, e.g., a lymph node removal or radiation therapy. In some embodiments, a subject is genetically predisposed to developing diseases or disorders of the lymphatic vasculature. In some embodiments, a subject is diagnosed with a disease or condition of the lymphatic vasculature such as, but not limited to lymphedema, obesity, inflammatory bowel disease/Crohn's disease, cellulitis, cancer, glaucoma, some forms of neural pathology, Hodgkin's Disease/Hodgkin's Lymphoma, Non-Hodgkin's Lymphoma, lymphadenitis, lymphangitis, and Lymphocytosis.

Lymphedema may be primary or secondary. Primary lymphedema can be present from birth (congenital lymphedema), may occur during puberty (lymphedema praecox), and is less often present later in life (lymphedema tarda). Secondary lymphedema is often a result of infection, especially dermatophytosis in the foot. In older persons, it may be due to malignant disease in the pelvis or groin and may follow surgical removal of lymph nodes and/or radiotherapy. Lymphedema may be complicated by infection (lymphangitis), which is manifested by chills, high fever, toxicity, and a red, hot, swollen leg. Lymphangitic streaks may be seen in the skin, and lymph nodes in the groin are usually enlarged and tender. These features differentiate lymphangitis from acute thrombophlebitis. Lymphedema patients are also prone to recurrent attacks of soft tissue bacterial infection (cellulites or erysipelas; the accompanying signs of infection are often blunted. These recurrent infections are the source of substantial morbidity and are difficult to prevent or eradicate. In the United States, the highest incidence of lymphedema is observed following breast cancer surgery, particularly among those who undergo radiation therapy following axillary lymphadenectomy. Among this population, 10-40% develop some degree of ipsilateral upper extremity lymphedema. Worldwide, 140-250 million cases of lymphedema are estimated to exist, with filariasis being the most common cause. Prevalence estimates of lymphedema, both in the United States and worldwide, are indirect, and likely reflect an underestimation of the burden of disease. The goal of conservative therapy is to eliminate protein stagnation and restore normal lymphatic circulation. These techniques are often cumbersome, uncomfortable, inconvenient, and time-consuming. Strict compliance is essential, and treatment lasts throughout the lifetime of the individual. Current treatment often includes careful hygiene and antimicrobial therapy. Patients often wear compression garments continuously during the day. Intermittent pneumatic pump compression therapy may also be instituted on an outpatient basis or in the home.

Causes of diseases, conditions, or injuries of the lymphatic vasculature are not intended to be limiting and can include any one or more of the following: genetic predisposition, disease, trauma, infections (bacterial, viral, fungal), sensitivity to non-infectious bacteria or toxins, allergies, transplant, cancer, exposure to toxins, congenital conditions, lifestyle choices, age, trauma (surgical or non-surgical), radiation exposure, and chemotherapy.

Methods of diagnosing disease and conditions of the lymphatic vasculature and methods for monitoring improvement in the symptoms of such disease, condition, and injuries (e.g., during and/or after treatment) include but are not limited to evaluating PF4 protein and/or RNA levels, e.g., from blood, plasma, or plasma exosomes, monitoring patient weight, limb swelling, subcutaneous fat content, overall patient health, prior medical history, evaluating genetic or molecular data indicating a genetic predisposition. Imaging approaches may include indirect radionuclide lymphoscintigraphy, near infrared lymphoscintigraphy, magnetic resonance lymphangiography (direct or indirect), and fluorescent lymphography. Supportive testing may include bioimpedance spectroscopy, measurement of excess limb volume and ultrasoography of the dermal lymphatic vessels.

### Pharmaceutical Compositions and Methods of Treatment

The compositions disclosed herein may include pharmaceutical compositions comprising an inhibitor of PF4, such as antibodies, small molecule inhibitors, and inhibitory nucleic acids.

Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, preservatives, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral or parenteral delivery. In some embodiments, compositions are formulated for site-specific administration, such as by injection into a specific tissue or organ, topical administration (e.g., by patch applied to the target tissue or target organ).

The therapeutic composition may include, in addition to an inhibitor of PF4, one or more additional active agents. By way of example, the one or more active agents may include an antibiotic, anti-inflammatory agent, a steroid, or a non-steroidal anti-inflammatory drug.

According to various aspects, an inhibitor of PF4, and optionally the one or more active or inactive agents may be present in the composition as particles or may be soluble. By way of example, in some embodiments, micro particles or microspheres may be employed, and/or nanoparticles may also be employed, e.g., by utilizing biodegradable polymers and lipids to form liposomes, dendrimers, micelles, or nanowafers as carriers for targeted delivery of the PF4 inhibitors. In some embodiments, polymeric implants may be used. By way of example, but not by way of limitation, in some embodiments, a therapeutic composition comprising PF4 inhibitor is applied to a patch and placed in contact with the target tissue.

In some embodiments, the composition formulated for administration comprises between 500 mg/ml and 1000 mg/ml of the inhibitor. In some embodiments, the composition formulated for administration comprises between 0.1 ng and 500 mg/ml of the inhibitor. In some embodiments, the compositions if formulated such that between 0.1 ng and 500 μg/ml of the inhibitor is administered to a subject.

Disclosed herein are methods of treating a disease or disorder of the lymphatic vasculature that comprises administering to a patient in need thereof, a pharmaceutical composition comprising an inhibitor of PF4.

In some embodiments, the composition is formulated for systemic delivery, and methods include administration via oral or parenteral delivery. In some embodiments, minimally invasive microneedles and/or iontophoresis may be used to administer the composition.

In some embodiments, the methods include administration of a therapeutic composition comprising a PF4 inhibitor to a subject by contacting the subject target tissue with the inhibitor, such as by targeted injection, or a patch embedded with the therapeutic composition and positioned to contact the target tissue.

In some embodiments, the methods include administration of the therapeutic compositions once per day; in some embodiments, the composition may be administered multiple times per day, e.g., at a frequency of one or two times per day, or at a frequency of three or four times per day or more. In some embodiments, the methods include administration of the composition once per week, once per month, or as symptoms dictate.

In some embodiments, the composition is administered at between 500 mg/ml and 1000 mg/ml of inhibitor; between 0.1 ng and 500 mg/ml of the inhibitor; or between about 0.1 ng and 500 μg/ml of the inhibitor.

In some embodiments, the treatment reduces, alleviates, prevents, or otherwise lessens the symptoms of the disease or condition more quickly than if no treatment is provided to a subject suffering the same or similar disease, condition or injury.

In some embodiments, improvements in the condition of the subject's lymphatic vasculature and overall health is observed more quickly than if no treatment is provided for the same or similar condition or disease.

By way of example, in some embodiments, improvements in the condition of the subject's lymphatic vasculature or overall health is observed within about 1 to about 3 days; within about 3 to about 5 days, or within about a week of the first administration. In some embodiments, improvements in the condition of the subject's lymphatic vasculature or overall health is observed within about 10 days, about 14 days or within about 1 month of the first administration. In some embodiments, improvements in the condition of the subject's lymphatic vasculature or overall health is observed within about 1-3 month, about 3-6 months or within about 1 year of the first administration.

### Embodiments

Some embodiments provide a method for identifying a disease or condition of the lymphatic vasculature in a subject in need thereof, the method comprising:
(a) determining a platelet factor 4 (PF4) level in a biological sample from the subject;
(b) comparing the determined PF4 level to a control PF4 level; wherein if the determined PF4 level is higher than the control PF4 level, identifying the subject as having the disease or condition of the lymphatic vasculature; and optionally
(c) administering to the subject a treatment for the disease or condition of the lymphatic vasculature.

In some embodiments, the subject has previously been diagnosed with obesity.

In some embodiments, the disease or condition of the lymphatic vasculature is lymphedema, lipedema, or lymphovascular disease.

In some embodiments, the biological sample comprises blood, blood plasma, or blood plasma exosomes.

The determined PF4 level can comprise a PF4 protein level or a PF4 RNA level.

In some embodiments, the subject is suffering from one or more of the following: swelling of part of an arm, leg, breast, or torso; a feeling of heaviness or tightness in part of an arm, leg, breast, or torso; restricted range of motion in an arm or leg; fibrosis; numbness or tingling in an arm or leg; infection; disproportionate fat below the waist.

In some embodiments, the subject has suffered lymphatic trauma, or has undergone a surgery, or has undergone a cancer treatment. In these embodiments, the cancer treatment may comprises one or more of: lymph node removal and radiation therapy.

In some embodiments, the subject has been diagnosed with a disease or condition of the lymphatic vasculature selected from the group consisting of obesity, inflammatory bowel disease/Crohn's disease, cellulitis, cancer, glaucoma, some forms of neural pathology, Hodgkin's Disease/Hodgkin's Lymphoma, Non-Hodgkin's Lymphoma, lymphadenitis, lymphangitis, lymphocytosis, lymphatic vascular malformation, chylous effusions, central conducting lymphatic anomaly, Kaposiform lymphangiomatosis, protein-losing enteropathy.

Also provided is method of treating a subject suspected of having, at risk of, or diagnosed with a disease or condition of the lymphatic vasculature, the method comprising:
(a) determining a platelet factor 4 (PF4) level in a biological sample from the subject;
(b) comparing the determined PF4 level to a control PF4 level; wherein if the determined PF4 level is higher than the control PF4 level, and
(c) treating the subject with a pharmaceutical composition comprising a molecule that specifically binds to and inhibits the function of PF4.

In some embodiments, the molecule that specifically binds to and inhibits the function of PF4 comprises an antibody.

In some embodiments, the subject has previously been diagnosed with obesity.

In some embodiments, the disease or condition of the lymphatic vasculature is lymphedema, lipedema, or lymphovascular disease.

In some embodiments, the biological samples comprises blood, blood plasma or blood plasma exosomes.

The determined PF4 level can comprise a PF4 protein level or a PF4 RNA level.

In some embodiments, the subject has suffered lymphatic trauma or has undergone a surgery, or has undergone a cancer treatment, e.g., one or more of: lymph node removal and radiation therapy.

In some embodiments, the subject has been diagnosed with a disease or condition of the lymphatic vasculature selected from the group consisting of obesity, inflammatory bowel disease/Crohn's disease, cellulitis, cancer, glaucoma, some forms of neural pathology, Hodgkin's Disease/Hodgkin's Lymphoma, Non-Hodgkin's Lymphoma, lymphadenitis, lymphangitis, lymphocytosis, lymphatic vascular malformation, chylous effusions, central conducting lymphatic anomaly, Kaposiform lymphangiomatosis, protein-losing enteropathy.

Also provided is method comprising:

(a) obtaining a biological sample from a subject exhibiting symptoms of lymphatic vascular dysfunction;

(b) contacting the biological sample with a reagent that detects PF4.

In some embodiments, the biological sample comprise a blood sample, a blood plasma sample or a blood plasma exosome sample.

In some embodiments, the reagent that detects PF4 detects PF4 protein, and comprises an antibody.

In some embodiments, the reagent that detects PF4 detects PF4 RNA and comprises a nucleic acid molecule that hybridizes to PF4 RNA or PF4 cDNA.

In some embodiments, the subject is exhibiting one or more the following symptoms: swelling of part of an arm, leg, breast, or torso; a feeling of heaviness or tightness in part of an arm, leg, breast, or torso; restricted range of motion in an arm or leg; fibrosis; numbness or tingling in an arm or leg; infection; disproportionate fat below the waist.

### Examples

The following examples are illustrative and should not be interpreted to limit the scope of the claimed subject matter.

In this investigation, mass spectrometry (MS) data were compared exosome proteomic signatures in normal, obese and lymphatic defective mouse models. A similar approach was used with plasma exosomes obtained from patients with various lymphatic disorders, with lipedema, and from obese and non-obese individuals without clinically overt lymphatic dysfunction. Platelet factor 4 (PF4) is reported herein as a plasma-circulating exosomal signature protein that could be used as a potential novel biomarker in the clinical setting to diagnose lymphatic vasculature dysfunction, and to distinguish these disorders from non-lymphatic-promoted obesity. Furthermore, we also found that PF4 levels were also increased in circulating exosomes from lipedema patients, a result that supports the prevailing hypothesis that the pathogenesis of this disease is, at least in part, lymphatic. However, exosomal PF4 levels are not associated with increased body weight, either in individuals with normal lymphatics or those with lymphatic-associated disorders.

### Materials and Methods

Mouse studies: Ob/Ob mice were obtained from the Jackson Laboratory (69). Prox1^{+/-} mice were generated and reported previously.

Exosomes purification and characterization: Gently mixed blood with EDTA was centrifuged at 500×g for 10 min at 10° C. Supernatant was centrifuged at 3000×g for 20 min at 10° C. Plasma was centrifuged at 12,000×g for 20 min at 10° C. to remove microvesicles and the supernatant was centrifuged at 100,000×g for 70 min at 10° C. The exosomes in the pellet fraction were washed with 20 ml of PBS and centrifuged at 100,000×g for 70 min at 10° C. The final exosome pellet was resuspended in 100 μl of PBS for analysis.

Human studies: Study subjects were recruited from the patient population of the Stanford Center for Lymphatic and Venous Disorders. The Administrative Panels for the Protection of Human Subjects of Stanford University (IRB 0000350) approved the protocols. Investigations were conducted according to the Declaration of Helsinki principles. Written consent was obtained from all recipients prior to inclusion in the studies. Phlebotomy was performed in the standard fashion, using a small gauge needle inserted into the brachiocephalic vein. 30 cc of blood were withdrawn in EDTA tubes, and the plasma was frozen at −80° C. for subsequent molecular analysis.

In order to be eligible for enrollment in this study, subjects were screened for the presence of lymphedema (primary or secondary), lipedema and lymphatic malformations. The diagnosis of lymphedema was based upon clinical evaluation, utilizing the criteria established by the International Society of Lymphology. The diagnosis of lipedema is based on commonly accepted clinical attributes. Normal control subjects were recruited from the same cardiovascular clinic as those with lymphatic pathologies; eligibility for enrollment included the absence of any clinically identifiable lymphatic pathology and the willingness to participate. In each subject cohort, the presence of obesity was defined as a BMI >30.

Mouse proteomic analysis: Proteins were dissolved using 8 M urea in 100 mM ammonium bicarbonate and 10 mM DTT. After reduction, cysteines were alkylated in 30 mM iodoacetamide. Proteins were then in-solution and digested with Lys-C (endoproteinase LysC, Wako Chemicals) in 4 M urea, followed by trypsinization (Trypsin Gold, Promega) in 2 M urea. Digestions were stopped by adding TFA and the digests were desalted using C₁₈ stage-tips.

Samples were analyzed by LC-MS/MS (Dionex 3000 coupled to Q-Exactive, Thermo Fisher). Peptides were separated by C-18 chromatography (inner diameter of 75 μm/3 μm particles, Nikkyo Technologies) using a gradient increasing from 1% B to 45% B in 135 min (A: 0.1% formic acid, B: acetonitrile in 0.1% formic acid). The peptides were electrosprayed (3.4 kV) into the mass spectrometer through a heated capillary at 320° C. and a S-Lens RF level of 60%. The mass spectrometer was operated in a data-dependent mode, with an automatic switch between the MS and MS/MS scans using a top 20 method (minimum AGC target 3E3) and a dynamic exclusion time of 45 sec. MS (300-1400 m/z) and MS/MS spectra were acquired with a resolution of 70,000 and 17,500 FWHM (200 m/z), respectively. Peptides were isolated using a 2 Th window and fragmented using higher-energy collisional dissociation (HCD) at 27% normalized collision energy. The ion target values were 5E5 for MS (100 ms maximum injection time) and 2E5 for MS/MS (60 ms maximum injection time).

Raw files were processed with MaxQuant (v 1.5.1.2) using the standard settings against a mouse protein database (UniProtKB/Swiss-Prot/TrEMBL, 43,539 sequences) supplemented with contaminants. Carbamidomethylation of cysteines was set as a fixed modification whereas oxidation of methionine and protein N-term acetylation as variable modifications. Minimal peptide length was set to 7 amino acids and a maximum of two tryptic missed cleavages were allowed. Results were filtered at 0.01 FDR (peptide and protein level). An arbitrary criteria of fold-change >0.5 or <−0.5 was used to define proteins as up or down regulated.

Human proteomic analysis: Proteins were dissolved using 8 M urea in 100 mM Tris-HCl pH 8.0. Protein concentration was determined using the Pierce® 660 nm Protein Assay (Bio-Rad) using BSA as standard. Then, samples (10-20 μg) were digested by means of the standard FASP protocol. Briefly, proteins were reduced and alkylated (15 mM TCEP, 30 mM CAA, 30 min in the dark, room temperature) and sequentially digested with Lys-C (Wako) (protein:enzyme ratio 1:50, o/n at RT) and trypsin (Promega) (protein:enzyme ratio 1:100, 6 h at 37° C.). Resulting peptides were desalted using C₁₈ stage-tips.

LC-MS/MS was done by coupling a nanoLC-Ultra 1D+ system (Eksigent) to an LTQ Orbitrap Velos mass spectrometer (Thermo Fisher Scientific) via a Nanospray Flex source (Thermo Fisher Scientific). Peptides were loaded into a trap column (NS-MP-10 BioSphere C18 5 μm, 20 mm length, Nanoseparations) for 10 min at a flow rate of 2.5 μl/min in 0.1% FA. Then peptides were transferred to an analytical column (ReproSil Pur C18-AQ 1.9 μm, 400 mm length and 0.075 mm ID) and separated using a 150 min linear gradient (buffer A: 4% ACN, 0.1% FA; buffer B: 100% ACN, 0.1% FA) at a flow rate of 250 nL/min. The gradient used was: 0-2 min 2% B, 3-133 min 30% B, 134-144 min 98% B, 145-150 min 2% B. The peptides were electro sprayed (1.8 kV) into the mass spectrometer with a PicoTip emitter (360/20 Tube OD/ID μm, tip ID 10 μm) (New Objective), a heated capillary temperature of 325° C. and S-Lens RF level of 60%. The mass spectrometer was operated in a data-dependent mode, with an automatic switch between MS and MS/MS scans using a top 20 method (threshold signal ≥800 counts and dynamic exclusion of 45 sec). MS spectra (350-1500 m/z) were acquired in the Orbitrap with a resolution of 60,000 FWHM (400 m/z). Peptides were isolated using a 1.5 Th window and fragmented using collision induced dissociation (CID) with linear ion trap read out at a NCE of 35% (0.25 Q-value and 10 ms activation time). The ion target values were 1E6 for MS (500 ms max injection time) and 5000 for MS/MS (100 ms max injection time).

Raw files were processed with MaxQuant (v 1.5.3.30) using the standard settings against a human protein database (UniProtKB/Swiss-Prot, December 2013, 20,584 sequences) supplemented with contaminants. Carbamidomethylation of cysteines was set as a fixed modification whereas oxidation of methionine and protein N-term acetylation as variable modifications. Minimal peptide length was set to 7 amino acids and a maximum of two tryptic missed cleavages were allowed. Results were filtered at 0.01 FDR (peptide and protein level). Afterwards, the “proteinGroups.txt” file was loaded in Prostar (v1.18) (72) using the intensity values for further statistical analysis. Briefly, proteins with less than 4 valid values in at least one experimental condition were filtered out. Then, a global normalization of log 2-transformed intensities across samples was performed using the LOESS function. Missing values were imputed using the algorithms SLSA (73) for partially observed values and DetQuantile for values missing on an entire condition. Differential analysis was done using the empirical Bayes statistics Limma. Proteins with a p.value <0.1 and a log 2 ratio >1 or <−1 were defined as regulated. The FDR was estimated to be up to 10% by Benjamini-Hochberg

ELISA: Total protein quantification in exosomes was performed using the BCA protein assay kit (Pierce). PF4 concentration in exosomes was quantified using a human PF4 ELISA kit (R&D) according to the manufacturer's instructions. The standards and the samples were run in duplicates. The results were read using a Synergy 2 plate reader. PF4 concentration was normalized with total protein content in exosomes.

Statistical Analysis: The analysis method for MS, demographic and co-morbidities was detailed in the text and table legends. All statistical analyses for ELISA were performed using GraphPad Prism 8.0. Data with parametric distribution were analyzed using unpaired Student t tests or 1-way analysis of variance (ANOVA); data with nonparametric distribution were analyzed by the Kruskal-Wallis test unless specified in the legends. All analyses with P values below 0.05 were considered statistically significant. Data represent mean value ±standard error of the mean (s.e.m.).

Study approval: All the mouse work was approved by the Northwestern University Animal Care and IACUC guidelines. Study subjects were recruited from the patient population of the Stanford Center for Lymphatic and Venous Disorders. The Administrative Panels for the Protection of Human Subjects of Stanford University (IRB 0000350) approved the protocols. Investigations were conducted according to the Declaration of Helsinki principles. Written consent was obtained from all recipients prior to inclusion in the studies.

### Results

### Exosomes Profiling in a Mouse Model of Lymphatic Malfunction

Detailed characterization of the lymphatic vasculature of E14.5 Prox1^{+/-} embryos showed that they displayed edema, indicating lymphatic dysfunction, but this phenotype resolved before birth. Detailed characterization of the lymphatic vasculature of E16.5 Prox1^{+/-} embryos and adult Prox1^{+/-} mice revealed mispatterning of the lymphatic vasculature; the most severely affected lymphatics were those of the intestine and mesentery, which were chyle-filled and ruptured. A low percentage of Prox1^{+/-} mice survive to adulthood and become significantly heavier than WT littermates at approximately 4 months of age, a consequence of the subtle leakage of lymph/chyle that promotes visceral accumulation of fat leading to obesity. Accordingly, we compared the protein profile of plasma-circulating exosomes from young non-obese (<3 months) and older obese (>5 months) Prox1^{+/-} mice, WT littermates and Ob/Ob mice (leptin receptor mutants) (31-33) that are severely obese but have a normal lymphatic vasculature (our unpublished results). It was reasoned that by comparing those groups biomarkers capable of distinguishing lymphatic malfunction (Prox1^{+/-} mice) from non-lymphatic-promoted obesity (i.e., Ob/Ob mice) and from WT mice could be identified.

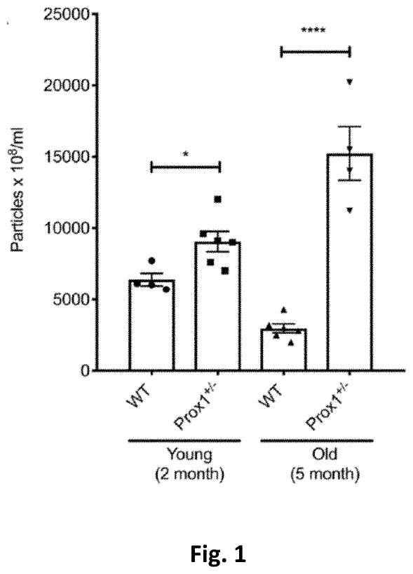

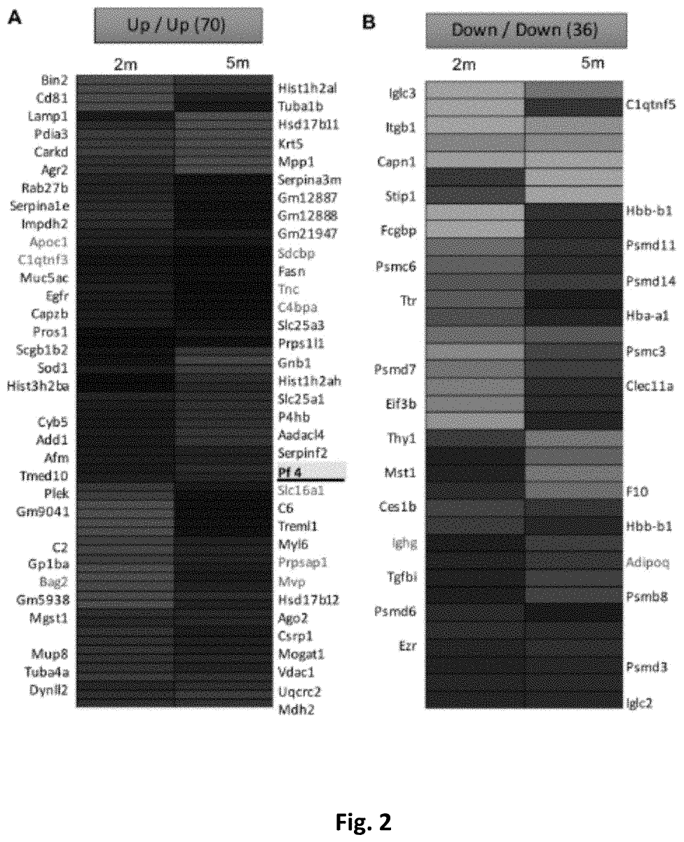

To isolate exosomes terminal bleeding was performed, and blood was collected by cardiac puncture. Circulating exosomes were purified from the isolated plasma using standard protocols (see Materials and Methods), and their presence and particle size was confirmed by Nanosight and by electron microscopy. Consistently, we found that in Prox1^{+/-} mice, the number of exosomes was higher than their age-matched littermate controls, either before or after the onset of obesity (FIG. 1). Next, exosomes were subjected to mass spectrometry (MS) to identify their protein cargo components. Due to the low survival rate of Prox1^{+/-} mice and the low plasma volume, the exosome yield was low. Therefore, for the MS analysis, plasma samples of animals with the same genotype were pooled. The proteomic signature of young (lean 3 month-old) and old (obese 5 month-old) Prox1^{+/-} mice and age-matched WT littermates was compared using a fold change cutoff of >0.5 or <−0.5 to identify the early changes that persists as disease develops. Using those criteria, 70 proteins were up-regulated in both young and old Prox1^{+/-} mice (FIG. 2 A) and 36 were down-regulated (FIG. 2 B). Important for the findings described below using the human samples, among the upregulated ones it was platelet factor 4 (PF4, FIG. 2 A). Pathway enrichment and protein-protein interaction network analysis (Encyclopedia of Genes and Genomes, KEGG) revealed that the upregulated proteins were mainly enriched in complement and coagulation cascades and systemic lupus erythematosus pathways (Table 1); in contrast, the downregulated ones were mainly identified in proteasome, Epstein-Barr virus infection and leukocyte trans-endothelial migration pathways (Table 2).

TABLE 1
KEGG pathways analysis of increased exosomal proteins in
young and old Prox1^{+/-} mice compared to age-matched WT mice.
KEGG Pathways

| | | | false |
|---|---|---|---|
| | | count in | discovery |
| pathway | Description | gene set | rate |
| mmu04610 | Complement and | 6 of 88 | 4.84E−05 |
| | coagulation cascades | | |
| mmu05322 | Systemic lupus erythematosus | 4 of 92 | 0.0123 |

TABLE 2
KEGG pathway analysis of decreased exosomal proteins in
young and old Prox1^{+/-} mice compared to age-matched WT mice.
KEGG Pathways

| | | | false |
|---|---|---|---|
| | | count in | discovery |
| pathway | description | gene set | rate |
| mmu03050 | Proteasome | 8 of 45 | 4.13E−14 |
| mmu05169 | Epstein-Barr virus infection | 7 of 205 | 8.27E−08 |
| mmu04670 | Leukocyte transendothelial | 3 of 115 | 0.0051 |
| | migration | | |

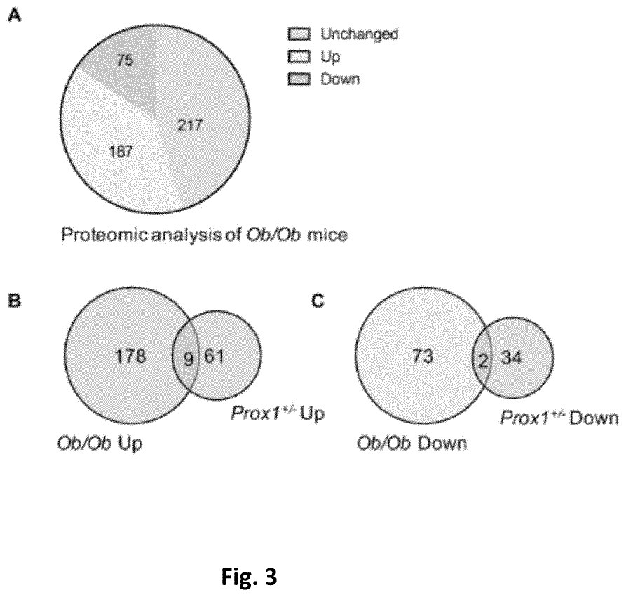

A similar MS analysis was them performed using pooled plasma from Ob/Ob and WT mice. Among the 479 proteins, 187 were increased and 75 were decreased in the Ob/Ob group (FIG. 3 A). To exclude proteins related to obesity, the Prox1^{+/-} mice dataset was compared with the ones from WT and Ob/Ob mice. 9 upregulated proteins and 2 downregulated proteins common to Prox1^{+/-} and Ob/Ob mice were identified, and narrowed the lymphatic specific signature in Prox1^{+/-} to 61 upregulated and 34 downregulated proteins (FIG. 3 B-C).

### Isolation and Characterization of Exosomes from Lymphedema Patients

To further validate and expand the animal model results described above, a similar analysis was performed with plasma-circulating exosomes isolated from patients with lymphatic dysfunction and from normal subjects. To do this an initial pilot experiment was performed; although the pilot study included a relatively limited number of subjects, the patient cohorts were generally well-matched by demographic variables (Table 3). The studied cohorts included lean and obese healthy subjects without overt lymphatic dysfunction, and patients with lymphatic disorders, including lean and obese subjects with secondary lymphedema, lean and obese subjects with lymphovascular disease, and lean and obese subjects with lipedema. As expected, all of the lymphatic disease cohorts were female-predominated (Table 3). Also, as anticipated, the category of lymphovascular disease, which reflects developmental and genetic diseases, is characterized by a significantly younger mean age.

TABLE 3
Demographics and disease characterization of the lymphatic subjects and normal controls

| | LYMPHEDEMA | LIPEDEMA | LYMPHOVASCULAR | CONTROL |
|---|---|---|---|---|
| | (N = 37) | (N = 15) | (N = 11) | (N = 12) |
| Age (years) | 58 ± 13 | 61 ± 13 | 41 ± 17** | 64 ± 14 |
| Female Gender no. (%) | 35 (95) | 15 (100) | 7 (64)* | 5 (14)* |
| Post-menopausal (%) | 7 (19) | 3 (20) | 3 (43) | 1 (8) |
| White | 26 (70) | 14 (93) | 10 (91) | 9 (75) |
| Black | 1 (3) | 1 (7) | 0 | 1 (8) |
| Asian | 2 (5) | 0 | 0 | 1 (8) |
| Other | 8 (22) | 0 | 1 (9) | 1 (9) |
| BMI | 34 ± 16 | 36 ± 9 | 33 ± 12 | 32 ± 7 |
| Obesity no. (%) | 14 (38) | 12 (80)* | 6 (55) | 7 (58) |
| Disease duration (years) | 15 ± 12 | 32 ± 19 ** | 20 ± 7** | — |
| Limbs Affected | | | | |
| Upper no. (%) | 8 (22) | — | 0 | — |
| Lower no. (%) | 28 (76) | 15 (100) | 7 (100) | — |
| Unilateral lower (%) | 4 (11) | — | 2 (29) | |
| Other (%) | 1 (3)+ | 1 (7) [symbol] | 0 | — |
| Lymphedema ISL Stage | | | | |
| Stage I | 1 (3) | — | — | — |
| Stage II | 31 (84) | — | 6 (86) | — |
| Stage III | 5 (13) | — | 1 (14) | — |
| Lipedema Stage | | | | |
| Stage I | | 4 (27) | | |
| Stage II | | 6 (40) | | |
| Stage III | | 5 (33) | | |
| Stage IV | | — | | |
| Lymphatic disease | | | | |
| etiology | | | | |
| Primary lymphedema | | — | 7 (64) | — |
| no. (%) | | | | |
| Secondary lymphedema | 37 (100) | — | — | — |
| no. (%) | | | | |
| Lipedema no. (%) | | 15 (100) | — | — |
| Lymphatic | | — | 3 (27) | — |
| malformation (%) | | | | |
| Lymphangiectasia (%) | | — | 1 (9) | — |
| Cancer-related no. (%) | | — | 18 (49) | — |

Kruskal-Wallace tests were performed for all continuous variables, and statistical comparisons were performed relative to lymphedema, unless indicated. Continuous variables were subjected to Mann-Whitney testing and discontinuous variables to the Fisher Exact test. Lymphatic disease etiology was not statistically examined. Unless indicated, the subject populations did not differ statistically. * p<0.05, **p<0.01, ***p<0>001, compared to lymphedema subjects; +pelvic lymphedema only; [symbol] 4-limb lipedema.

Initially, the focus was on the molecular differences between normal individuals and lymphatic patients, and therefore did not segregate individuals by BMI. Following exosome purification and MS analysis, 4 samples pooled from 8 normal subjects without overt lymphatic dysfunction, 8 pooled samples from 15 secondary lymphedema patients, 3 samples from 3 lymphovascular patients and 8 samples from 8 lipedema patients were profiled. Proteins with a p.value <0.1 and a log 2 ratio >1 or <−1 were considered to be differentially regulated. From this analysis, 13 increased and 14 decreased proteins were identified in secondary lymphedema patients (Table 4-5), 38 increased and 55 decreased proteins were identified in lymphovascular disease patients (Table 6-7, only the top 50 decreased proteins are shown due to space limits) and 19 increased and 35 decreased proteins were identified in lipedema patients (Table 8-9). Of interest, and as shown in Tables 4, 6 and 8, among this list of upregulated proteins, platelet factor 4 (PF4) was the only one whose levels were elevated (when compared with normal controls) in samples from secondary lymphedema, lymphovascular disease (including primary lymphedema), lipedema patients and also in Prox1^{+/-} mice (FIG. 2 A). Platelet factor 4 is a protein released from platelets and is known to be able to inhibit angiogenesis and to promote innate immune responses, making this protein an interesting target for inflammation. PF4 bound to surface glycosaminoglycans on platelets, monocytes and endothelial cells is also an immunogenic target in prothrombotic disorders. The concentration of PF4 in serum after platelet activation is a thousand-fold higher than in plasma (35-38).

TABLE 4
List of increased proteins in secondary lymphedema
patients compared to healthy individuals

| Protein names | Gene names | LogFC |
|---|---|---|
| Platelet factor 4;Platelet factor 4, short form | PF4 | 3.22 |
| Pregnancy zone protein | PZP | 1.75 |
| Putative V-set and immunoglobulin domain- | IGHV4OR15-8 | 1.55 |
| containing-like protein IGHV4OR15-8 | | |
| Proto-oncogene tyrosine-protein kinase Src | SRC | 1.36 |
| Calcium-independent phospholipase A2-gamma | PNPLA8 | 1.32 |
| Tubulin alpha-4A chain | TUBA4A | 1.25 |
| Pleckstrin | PLEK | 1.18 |
| Ig kappa chain V-II region Cum | | 1.15 |
| Complement C2; Complement C2b | C2 | 1.05 |
| fragment; Complement C2a fragment | | |
| Tubulin beta-1 chain | TUBB1 | 1.05 |
| Glyceraldehyde-3-phosphate dehydrogenase | GAPDH | 1.02 |
| Serum amyloid A-4 protein | SAA4 | 1.02 |
| Haptoglobin; Haptoglobin alpha | HP | 1.01 |
| chain; Haptoglobin beta chain | | |

TABLE 5
List of decreased proteins in secondary lymphedema
patients compared to healthy individuals.

| Protein names | Gene names | LogFC |
|---|---|---|
| Reelin | RELN | −2.30 |
| Extracellular matrix protein 1 | ECM1 | −2.01 |
| von Willebrand factor; von Willebrand antigen 2 | VWF | −1.84 |
| Tenascin | TNC | −1.72 |
| Coagulation factor VIII; Factor VIIIa heavy chain, | F8 | −1.70 |
| 200 kDa isoform; Factor VIIIa heavy chain, 92 | | |
| kDa isoform; Factor VIII B chain; Factor Villa | | |
| light chain | | |
| Ig kappa chain V-I region AG | | −1.53 |
| T-complex protein 1 subunit alpha | TCP1 | −1.47 |
| Band 3 anion transport protein | SLC4A1 | −1.27 |
| Ig heavy chain V-II region SESS | | −1.27 |
| Sushi, von Willebrand factor type A, EGF and | SVEP1 | −1.25 |
| pentraxin domain-containing protein 1 | | |
| Moesin | MSN | −1.25 |
| Ig alpha-2 chain C region | IGHA2 | −1.21 |
| Ig kappa chain V-I region AU | | −1.17 |
| Catalase | CAT | −1.16 |

TABLE 6
List of increased proteins in lymphovascular
disease patients compared to healthy individuals

| Protein names | Gene names | LogFC |
|---|---|---|
| Ig kappa chain V-IV region; | IGKV4-1 | 8.14 |
| Ig kappa chain VW region JI | | |
| Pregnancy zone protein | PZP | 3.46 |
| Ig kappa chain V-III region IARC/BL41 | | 3.03 |
| Ig lambda chain V-V region DEL | | 2.51 |
| Platelet factor 4; Platelet factor 4, short form | PF4 | 2.41 |
| C-reactive protein; C-reactive protein(1-205) | CRP | 2.27 |
| Ig kappa chain V-I region Mev | | 2.21 |
| Ig kappa chain V-III region NG9 | | 2.16 |
| Alcohol dehydrogenase class-3 | ADH5 | 1.99 |
| Peroxiredoxin-2 | PRDX2 | 1.86 |
| Desmoglein-1 | DSG1 | 1.81 |
| Ig kappa chain V-III region Ti | | 1.80 |
| Ig heavy chain V-III region TIL | | 1.79 |
| Ig lambda chain V-III region SH | | 1.77 |
| Ig heavy chain V-III region 23 | IGHV3-23 | 1.71 |
| Ig kappa chain V-II region MIL | | 1.56 |
| Complement C2; Complement C2b | C2 | 1.51 |
| fragment; Complement C2a | | |
| Ig lambda chain V-VI region WLT; | | 1.48 |
| Ig lambda chain V-VI region EB4 | | |
| Complement factor D | CFD | 1.45 |
| Proto-oncogene tyrosine-protein kinase Src | SRC | 1.34 |
| Ig lambda chain V-II region BUR | | 1.32 |
| Fibulin-5 | FBLN5 | 1.28 |
| Clathrin heavy chain 1 | CLTC | 1.24 |
| Ig kappa chain V-I region Wes | | 1.22 |
| Ig lambda chain V-I region WAH | | 1.21 |
| Ig lambda chain V region 4A | | 1.20 |
| Ig kappa chain V-II region TEW | | 1.20 |
| Fetuin-B | FETUB | 1.19 |
| Ig heavy chain V-I region V35 | | 1.14 |
| Complement factor H-related protein 1 | CFHR1 | 1.14 |
| T-complex protein 1 subunit gamma | CCT3 | 1.12 |
| Ig kappa chain V-I region BAN | | 1.11 |
| Complement C4-A; | C4A | 1.11 |
| Complement C4 beta chain; | | |
| Complement C4-A alpha chain; | | |
| C4a anaphylatoxin; | | |
| C4b-A; C4d-A; | | |
| Complement C4 gamma chain | | |
| Junction plakoglobin | JUP | 1.11 |
| Ig lambda chain V-I region NIG-64; | | 1.11 |
| Ig lambda chain V-I region BL2 | | |
| Ig lambda chain V-IV region MOL | | 1.10 |
| Ig lambda chain V-I region NEW | | 1.06 |
| Ig heavy chain V-III region HIL | | 1.02 |

TABLE 7
List of top 50 decreased proteins in lymphovascular
disease patients compared to healthy individuals

| Protein names | Gene names | LogFC |
|---|---|---|
| Coagulation factor VIII; Factor VIIIa | F8 | −3.45 |
| heavy chain, 200 kDa isoform; Factor | | |
| VIIIa heavy chain, 92 kDa isoform; Factor | | |
| VIII B chain;Factor VIIIa light chain | | |
| Ficolin-3 | FCN3 | −3.43 |
| Integrin alpha-6; Integrin alpha-6 heavy | ITGA6 | −3.08 |
| chain; Integrin alpha-6 light | | |
| chain; Processed integrin alpha-6 | | |
| Mannan-binding lectin serine protease | MASP2 | −2.96 |
| 2; Mannan-binding lectin serine protease | | |
| 2 A chain; Mannan-binding lectin serine | | |
| protease 2 B chain | | |
| Tubulin beta-1 chain | TUBB1 | −2.63 |
| Erythrocyte band 7 integral | STOM | −2.47 |
| membrane protein | | |
| Tenascin-X | TNXB | −2.46 |
| Mannan-binding lectin serine protease | MASP1 | −2.42 |
| 1; Mannan-binding lectin serine protease | | |
| 1 heavy chain; Mannan-binding lectin | | |
| serine protease 1 light chain | | |
| Ig alpha-2 chain C region | IGHA2 | −2.37 |
| CD9 antigen | CD9 | −2.37 |
| Actin, alpha skeletal muscle; | ACTAl; ACTC1; | −2.37 |
| Actin, alpha cardiac muscle 1; | ACTG2; ACTA2 | |
| Actin, gamma-enteric smooth muscle; | | |
| Actin, aortic smooth muscle | | |
| Talin-1 | TLN1 | −2.35 |
| Filamin-A | FLNA | −2.33 |
| 14-3-3 protein zeta/delta | YWHAZ | −1.99 |
| Band 3 anion transport protein | SLC4A1 | −1.94 |
| Vinculin | VCL | −1.93 |
| Integrin alpha-IIb; Integrin alpha-IIb | ITGA2B | −1.90 |
| heavy chain; Integrin alpha-IIb light | | |
| chain, form 1; Integrin alpha-IIb light | | |
| chain, form 2 | | |
| Tenascin | TNC | −1.88 |
| Apolipoprotein C-III | APOC3 | −1.85 |
| von Willebrand factor; | VWF | −1.82 |
| von Willebrand antigen 2 | | |
| Integrin beta-3 | ITGB3 | −1.77 |
| Catalase | CAT | −1.73 |
| A disintegrin and metalloproteinase | ADAMTS13 | −1.68 |
| with thrombospondin motifs 13 | | |
| Tubulin alpha-4A chain | TUBA4A | −1.65 |
| Ubiquitin-60S ribosomal protein L40; | UBA52; RPS27A; | −1.65 |
| Ubiquitin; 60S ribosomal protein L40; | UBB; UBC | |
| Ubiquitin-40S ribosomal protein S27a; | | |
| Ubiquitin; 40S ribosomal protein S27a; | | |
| Polyubiquitin-B; | | |
| Ubiquitin; Polyubiquitin-C; Ubiquitin | | |
| ADP-ribosylation factor 1; | ARF1; ARF3; | −1.63 |
| ADP-ribosylation factor 3; | ARF5; ARF4 | |
| ADP-ribosylation factor 5; | | |
| ADP-ribosylation factor 4 | | |
| Apolipoprotein F | APOF | −1.62 |
| Hemoglobin subunit alpha | HBA1 | −1.62 |
| Integrin beta-1 | ITGB1 | −1.59 |
| Extracellular matrix protein 1 | ECM1 | −1.57 |
| Carbonic anhydrase 1 | CA1 | −1.57 |
| Actin, cytoplasmic 2; Actin, | ACTG1 | −1.51 |
| cytoplasmic 2, N-terminally processed | | |
| Apolipoprotein B-100; | APOB | −1.49 |
| Apolipoprotein B-48 | | |
| Fermitin family homolog 3 | FERMT3 | −1.46 |
| Apolipoprotein C-I; | APOC1 | −1.45 |
| Truncated apolipoprotein C-I | | |
| Ras suppressor protein 1 | RSU1 | −1.44 |
| Serum amyloid A-1 protein; | SAA1 | −1.43 |
| Amyloid protein A; | | |
| Serum amyloid protein A (2-104); | | |
| Serum amyloid protein A (3-104); | | |
| Serum amyloid protein A (2-103); | | |
| Serum amyloid protein A (2-102); | | |
| Serum amyloid protein A (4-101) | | |
| Hemoglobin subunit delta | HBD | −1.41 |
| Alpha-actinin-1 | ACTN1 | −1.41 |
| Moesin | MSN | −1.38 |
| Beta-parvin | PARVB | −1.37 |
| Adenylyl cyclase-associated protein 1 | CAP1 | −1.35 |
| Basement membrane-specific | HSPG2 | −1.33 |
| heparan sulfate proteoglycan core | | |
| protein; Endorepellin; LG3 peptide | | |
| Hemoglobin subunit beta; | HBB | −1.28 |
| LVV-hemorphin-7; Spinorphin | | |
| Coagulation factor XIII B chain | F13B | −1.27 |
| Ig heavy chain V-III region GA | | −1.24 |
| Ficolin-2 | FCN2 | −1.24 |
| HLA class I histocompatibility antigen, | HLA-A | −1.18 |
| A-68 alpha chain | | |
| Apolipoprotein C-II; | APOC2 | −1.18 |
| Proapolipoprotein C-II | | |
| Alpha-2-HS-glycoprotein; | AHSG | −1.13 |
| Alpha-2-HS-glycoprotein chain A; | | |
| Alpha-2-HS-glycoprotein chain B | | |

TABLE 8
List of increased proteins in lipedema patients compared to healthy individuals

| Protein names | Gene names | LogFC |
|---|---|---|
| Ig kappa chain V-IV region; | IGKV4-1 | 7.29 |
| Ig kappa chain V-IV region JI | | |
| Platelet glycoprotein Ib beta chain | GP1BB | 2.61 |
| Myosin-9 | MYH9 | 2.32 |
| Pleckstrin | PLEK | 2.13 |
| Myosin light polypeptide 6 | MYL6 | 2.12 |
| Ig kappa chain V-III region IARC/BL41 | | 2.12 |
| Pregnancy zone protein | PZP | 1.66 |
| Multimerin-1; | MMRN1 | 1.44 |
| Platelet glycoprotein Ia*; | | |
| 155 kDa platelet multimerin | | |
| Tubulin alpha-4A chain | TUBA4A | 1.39 |
| Alpha-actinin-1 | ACTN1 | 1.37 |
| Transgelin-2 | TAGLN2 | 1.36 |
| Platelet factor 4; | PF4 | 1.26 |
| Platelet factor 4, short form | | |
| Actin, alpha skeletal muscle; | ACTA1; ACTC1; | 1.25 |
| Actin, alpha cardiac muscle 1; | ACTG2; ACTA2 | |
| Actin, gamma-enteric smooth muscle; | | |
| Actin, aortic smooth muscle | | |
| Peptidyl-prolyl cis-trans isomerase A; | PPIA | 1.19 |
| Peptidyl-prolyl cis-trans isomerase A, | | |
| N-terminally processed | | |
| Putative V-set and immunoglobulin | IGHV4OR15-8 | 1.11 |
| domain-containing-like protein | | |
| IGHV4OR15-8 | | |
| Profilin-1 | PFN1 | 1.10 |
| Tropomyosin alpha-4 chain | TPM4 | 1.06 |
| Complement factor H-related protein 1 | CFHR1 | 1.04 |
| Complement C4-A; | C4A | 1.04 |
| Complement C4 beta chain; | | |
| Complement C4-A alpha chain; | | |
| C4a anaphylatoxin; | | |
| C4b-A; C4d-A; | | |
| Complement C4 gamma chain | | |

TABLE 9
List of decreased proteins in lipedema patients compared to healthy individuals

| Protein names | Gene names | LogFC |
|---|---|---|
| Reelin | RELN | −2.58 |
| Spermatid-associated protein | SPERT | −2.34 |
| Cartilage acidic protein 1 | CRTAC1 | −2.26 |
| Clathrin heavy chain 1 | CLTC | −2.23 |
| Collagen alpha-3(VI) chain | COL6A3 | −2.17 |
| Ig kappa chain V-I region HK101 | | −1.71 |
| Hemoglobin subunit delta | HBD | −1.62 |
| ADP-ribosylation factor 1; | ARF1; ARF3; | −1.62 |
| ADP-ribosylation factor 3; | ARF5; ARF4 | |
| ADP-ribosylation factor 5; | | |
| ADP-ribosylation factor 4 | | |
| Apolipoprotein F | APOF | −1.57 |
| Solute carrier family 2, facilitated | SLC2A1 | −1.53 |
| glucose transporter member 1 | | |
| Transferrin receptor protein 1; | TFRC | −1.52 |
| Transferrin receptor protein 1, serum form | | |
| Ig kappa chain V-I region AU | | −1.48 |
| Integrin alpha-6; | ITGA6 | −1.47 |
| Integrin alpha-6 heavy chain; | | |
| Integrin alpha-6 light chain; | | |
| Processed integrin alpha-6 | | |
| Band 3 anion transport protein | SLC4A1 | −1.46 |
| Methionine adenosyltransferase | MAT2B | −1.45 |
| 2 subunit beta | | |
| Tenascin-X | TNXB | −1.43 |
| Syntenin-1 | SDCBP | −1.40 |
| Ig lambda-7 chain C region | IGLC7 | −1.35 |
| Ras suppressor protein 1 | RSU1 | −1.35 |
| Apolipoprotein B-100; | APOB | −1.34 |
| Apolipoprotein B-48 | | |
| Lipopolysaccharide-binding protein | LBP | −1.31 |
| Carbonic anhydrase 1 | CA1 | −1.30 |
| Coagulation factor IX; | F9 | −1.28 |
| Coagulation factor IXa light chain; | | |
| Coagulation factor IXa heavy chain | | |
| Carboxypeptidase B2 | CPB2 | −1.26 |
| CD9 antigen | CD9 | −1.26 |
| Catalase | CAT | −1.24 |
| Coagulation factor XI; | F11 | −1.24 |
| Coagulation factor XIa heavy chain; | | |
| Coagulation factor XIa light chain | | |
| Ig lambda chain V-I region NIG-64; | | −1.22 |
| Ig lambda chain V-I region BL2 | | |
| Cholesteryl ester transfer protein | CETP | −1.18 |
| Cholinesterase | BCHE | −1.18 |
| Zinc finger protein with | ZKSCAN2 | −1.11 |
| KRAB and SCAN domains 2 | | |
| Beta-Ala-His dipeptidase | CNDP1 | −1.09 |
| Transforming growth factor- | TGFBI | −1.08 |
| beta-induced protein ig-h3 | | |
| Erythrocyte band 7 integral membrane protein | STOM | −1.05 |
| Ig kappa chain V-II region FR | | −1.02 |

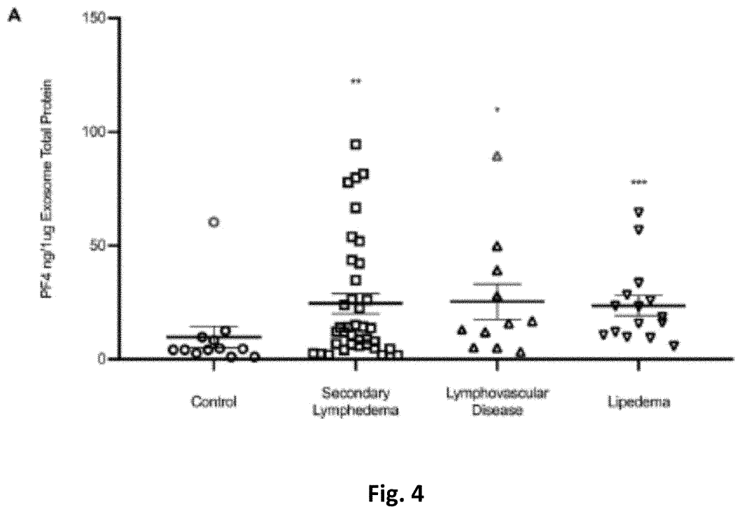

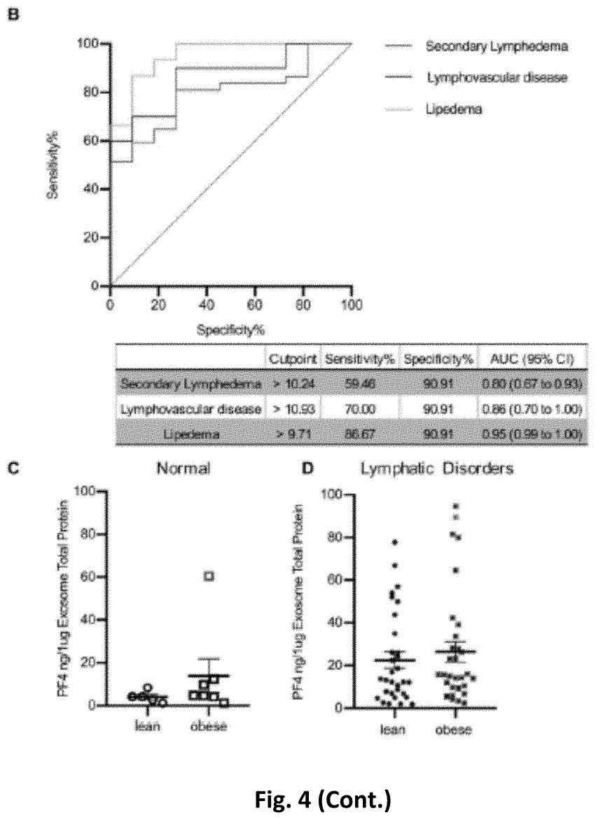

Next, these initial results were further validated using a human PF4 ELISA assay. The exosomes protein cargo from 12 normal subjects, 37 lymphedema patients, 11 lymphovascular disease patients and 15 lipedema patients was analyzed (protein content was normalized for each sample). This ELISA analysis validated the MS results described above, as PF4 levels were elevated in all patients except one (FIG. 4 A); as determined by Grubb's test (39), this single non-lymphedema obese patient with very high PF4 levels was an outlier with a history of inflammatory bowel disease; therefore, it was removed from this graph (FIG. 4 A). Although the pathophysiology of IBD remains unknown, alterations in the intestinal lymphatics are becoming accepted features of IBD, particularly in Crohn's disease subjects (40-42). To evaluate the diagnostic power of PF4 for lymphatic alterations, a receiver operating characteristic (ROC) curve analysis was performed. As shown in FIG. 4 B, the areas under the ROC Curve (AUCs) were 0.80 (95% confidence interval (CI) 0.67 to 0.93), 0.86 (95% CI: 0.70 to 1.00) and 0.95 (95% CI: 0.99 to 1.00) for secondary lymphedema, lymphovascular disease and lipedema patients, respectively. At the corresponding optimal cutoff values, the sensitivities and specificities of PF4 to predict secondary lymphedema reached 59.46% and 90.91%, for lymphovascular reached 70.00% and for lipedema reached 86.67% and 90.91%, respectively.

To eliminate the likelihood that co-morbidities might be responsible for the observed differences in PF4 levels, the distribution of co-morbidities among the subjects in each of the enrolled cohorts was examined (Table 10). The only significant differences observed were that of a reduced incidence of cancer in the lipedema cohort when compared to those with lymphedema, and increased hypertension and musculoskeletal disease in the control group when compared to those with lymphedema. Of note, no identified platelet disorder was listed among these patients. In parallel with the human clinical observations, PF4 was also upregulated in both young and old Prox1^{+/-} mice (FIG. 2 B) but not in Ob/Ob mice. These findings suggest that PF4 could be a novel biomarker for lymphatic disorders.

TABLE 10
Comorbidities (all in %)

| | LYMPHEDEMA | LIPEDEMA | LYMPHOVASCULAR | CONTROL |
|---|---|---|---|---|
| | (N = 37) | (N = 15) | (N = 11) | (N = 12) |
| Cancer history | 18 (49) | 2 (13)* | 2 (18) | 4 (33) |
| Venous disease | 5 (14) | 5 (33) | 0 | 1 (8) |
| Cellulitis history | 12 (32) | 2 (13) | 3 (27) | 1 (8) |
| Hypertension | 13 (35) | 5 (33) | 0 | 9 (75)* |
| Dyslipidemia | 10 (27) | 4 (27) | 2 (18) | 7 (58) |
| Cardiac Arrhythmia | 3 (8) | 0 | 0 | 4 (33) |
| Coronary artery disease | 0 | 2 (13) | 2 (18) | 2 (17) |
| Congestive heart failure | 0 | 2 (13) | 0 | 1 (8) |
| Hypothyroidism | 10 (27) | 2 | 0 | 1 (8) |
| Glucose | 5 (14) | 2 (13) | 0 | 1 (8) |
| intolerance/ | | | | |
| Diabetes mellitus | | | | |
| Skin condition | 6 (16) | 0 | 0 | 3 (25) |
| Lung disease | 4 (11) | 3 (20) | 0 | 1 (8) |
| Pulmonary | 0 | 1 (7) | 1 (9) | 0 |
| hypertension | | | | |
| Anemia/ | 5 (11) | 0 | 2 (18) | 3 (25) |
| Hematologic disease | | | | |
| Hyperparathyroidism | 2 (5) | 0 | 0 | 0 |
| Migraine | 4 (11) | 0 | 0 | 1 (8) |
| Irritable bowel | 2 (5) | 0 | 3 (27) | 0 |
| syndrome | | | | |
| Ulcerative colitis | 1 (3) | 1 (7) | 0 | 2 (17) |
| Musculoskeletal | 11 (30) | 8 (53) | 0 | 11 (92)* |
| disease | | | | |
| Gynecological | 5 (14) | 4 (27) | 0 | 1 (8) |
| diseases | | | | |
| Obstructive sleep apnea | 4 (11) | 0 | 1 (9) | 0 |
| Anxiety/Depression | 13 (35) | 7 (47) | 3 (27) | 0* |
| Vitamin D deficiency | 3 (8) | 0 | 0 | 0 |
| Gastric ulcer | 7 (19) | 1 (7) | 0 | 0 |
| Gastrointestinal | 4 (11) | 4 (27) | 1 (9) | 0 |
| disorders | | | | |
| Allergy | 4 (11) | 0 | 0 | 4 (33) |
| Nephrolithiasis/ | 2 (5) | 1 (7) | 2 (18) | 1 (8) |
| hydronephrosis | | | | |
| Neuropathy | 8 (22) | 0 | 1 (9) | 1 (8) |
| Renal disease | 2 (5) | 0 | 1 (9) | 1 (8) |
| Structural | 4 (11) | 0 | 1 (9) | 0 |
| Cardiovascular disease | | | | |
| Macular degeneration | 1 (3) | 1 (7) | 0 | 0 |
| Addison’s disease | 1 (3) | 0 | 0 | 0 |
| Esophageal varices | 1 (3) | 0 | 0 | 0 |
| Pleural effusion | 2 (5) | 0 | 1 (9) | 0 |
| Hypogammaglobulinemia | 1 (3) | 0 | 0 | 0 |
| Deep vein | 0 | 3 (20) | 0 | 0 |
| thrombosis by history | | | | |
| Glaucoma | 0 | 1 (7) | 0 | 0 |
| Sarcoidosis | 0 | 1 (7) | 0 | 0 |
| Skin cancer | 0 | 1 (7) | 1 (9) | 0 |
| Cardiomyopathy | 0 | 0 | 0 | 2 (17) |

All statistical comparisons were with lymphedema and utilized the Fisher Exact test. Unless, designated, there were no statistically significant differences.
*p <0.05 compared to lymphedema.

Then, to explore if PF4 can distinguish normal lean and obese human subjects (not symptomatic lymphatic malfunction), from those with lymphatic disorders, we further separated lean and obese normal and lymphatic-affected individuals. As shown in FIG. 4C-D, the PF4 level is not statistically different in lean or obese normal or affected subjects. This suggests that PF4 can be used as a biomarker capable to distinguish normal subjects from those with lymphatic defects independent of the presence or absence of obesity.

Lymphedema is a devastating disease that lacks early diagnostic tools and readily available pharmacological interventions. Current accurate diagnosis of early or subclinical disease often relies upon sophisticated imaging techniques, which can be relatively invasive. Less invasive screening tools are not yet available. Although the more advanced stages of lymphedema can be clinically diagnosed, subtle, early, and subclinical disease can be elusive. Furthermore, with the recent surge of newly identified functional roles for the lymphatic vasculature in a variety of normal and pathological conditions (e.g., obesity, cardiovascular disease and neurodegenerative disorders), it is possible that individuals with any of those pathologies might be grossly asymptomatic for any of the typical features of lymphatic dysfunction. In such circumstances, the ready availability of reliable biomarkers could play a defining role in the screening and diagnosis of more subtle forms of lymphatic-associated defects.

The analysis described above, using mouse models and individuals with a variety of lymphatic pathologies, identified PF4 as a diagnostic marker for lymphatic-promoted disorders. The levels of PF4 were increased in both young and old Prox1^{+/-} mice (before and after the onset of obesity), as well as in lymphedema, lipedema and patients with heritable developmental diseases of the lymphatics. PF4, also called CXCL4, is a chemokine that is packaged in platelet alpha-granules and is secreted upon activation during inflammation and wound healing. Although this study does not identify the cell of origin of exosomal PF4 or the mechanism underlying exosomal PF4 secretion, it is possible to speculate that structurally and functionally defective lymphatics are responsible for mediating such signaling. Besides regulating hemostasis and thrombosis, platelets also play an important developmental role in the separation of the blood and lymphatic vascular networks. Platelets are activated by lymphatic endothelial cells to form a plug at the level of the lymphovenous valve, the structure where the central lymphatic vasculature connects to the blood vascular system. In mice, the failure to form such a platelet plug results in the reflux of blood into the lymphatic vessels and these mice develop lymphedema. It has been reported that PF4 is also increased in a mouse model of acute surgical lymphedema detected by cDNA microarray analysis. Prior studies suggested that PF4 inhibits angiogenesis in vivo and in vitro. For example, PF4 inhibits FGF2 and VEGF signaling through heparin-dependent and -independent mechanisms. However, whether increased exosomal PF4 inhibit lymphangiogenesis in vivo is not clear.

A few lines of evidence support the hypothesis that PF4 might play a role in lymphedema. It has been shown that PF4 induces chemotaxis of T lymphocytes and upregulates T helper 2 (Th2) cytokines in a CXCR3 dependent manner. This is relevant because T cells, including Th2 cells, are known to infiltrate lymphedematous tissue and play a role in inflammation, fibrosis and lymphangiogenesis. Blocking Th2 differentiation decreases fibrosis, improves lymphatic function and delays the progression of lymphedema. The elevated levels of PF4 detected in plasma-circulating exosomes of affected individuals might contribute to the recruitment and stimulation of Th2 cells in lymphedema patients. Moreover, it has been also reported that blood plasma PF4 levels are increased in patients with Crohn's disease, a disorder that has been recently shown to feature lymphatic alterations. The elevated platelet count appears to correlate with the presence of immature platelets in blood, that might play a role in predisposing IBD patients to thrombus development. It could be speculated that the increased levels of PF4 in Crohn's disease could also, at least partially, be the consequence of the associated alterations in the mesenteric lymphatic vasculature. In addition, lipopolysaccharides (LPS), which trigger pro-inflammatory responses in endothelial cells, increase PF4 levels and cell permeability by reducing tight junction proteins in cultured human umbilical vein endothelial cells (HUVEC). The authors suggested that the effect of LPS on cell permeability is mediated by PF4, since it can be abolished by PF4 neutralizing antibodies, and PF4 itself decreases tight junction proteins and promotes cell permeability. Potentially, it could be speculated that PF4 might increase blood vessel permeability and reduce lymphangiogenesis as contributing factors in lymphatic diseases. Supporting this hypothesis, pathological alterations of leukotriene biology have been observed in both murine and human lymphedema, with evidence of anti-lymphangiogenic concentrations of leukotriene B₄ (LTB₄) in these individuals. It is notable that LTB₄ also induced endothelial cell permeability in vivo (67). Future investigations of the role of PF4 in lymphatic dysfunction should encompass exploration of the relationship of PF4 to leukotriene-mediated effects in the pathogenesis of lymphedema and lymphatic-related disorders.

Some studies have suggested a close association of excessive fat accumulation with lymphatic dysfunction. It has previously shown that Prox1^{+/-} mice with defective lymphatics develop adult-onset obesity, likely a consequence of chyle leakage. Comparison of the exosome protein profile between Prox1^{+/-} and Ob/Ob mice demonstrated substantial differences and, specifically, PF4 was not increased in Ob/Ob mice. These data suggest that PF4 levels might also be useful to identify obese individuals in which at least some of the underlying pathogenesis of excessive fat accumulation could be subtle and asymptomatic lymphatic leakage. Finally, the results support the prevailing hypothesis that in lipedema, lymphatic dysfunction plays a role in the pathogenesis of the disease, as has previously been suggested on the basis of imaging attributes.

### REFERENCES

1. Escobedo N, Oliver G. Lymphangiogenesis: origin, specification, and cell fate determination. Annu Rev Cell Dev Biol. 2016; 32(1):annurev-cellbio-111315-124944.
2. Rockson S G. Lymphedema. Am J Med. 2001; 110(4):288-295.
3. Witte C L. Pumps and lymphedema. Lymphology. 2001; 34(4):150-151.
4. Greene A K, Maclellan R A. Obesity-induced upper extremity lymphedema. Plast Reconstr Surg Glob Open. 2013; 1(7):e59.
5. Wang Y, Oliver G. Current views on the function of the lymphatic vasculature in health and disease. Genes Dev. 2010; 24(19):2115-2126.
6. Tavakkolizadeh A, Wolfe K Q, Kangesu L. Cutaneous lymphatic malformation with secondary fat hypertrophy. Br J Plast Surg. 2001; 54(4):367-369.
7. Schirger A, Harrison E G, Janes J M. Idiopathic lymphedema. JAMA. 1962; 182(1):14.
8. Pond C M. Adipose tissue and the immune system. Prostaglandins, Leukot Essent Fat Acids. 2005; 73(1):17-30.
9. Rosen E D. The molecular control of adipogenesis, with special reference to lymphatic pathology. Ann N Y Acad Sci. 2002; 979(1): 143-158.
10. Lohrmann C, Foeldi E, Langer M. MR imaging of the lymphatic system in patients with lipedema and lipo-lymphedema. Microvasc Res. 2009; 77(3):335-339.
11. Bilancini S, Lucchi M, Tucci S, Eleuteri P. Functional lymphatic alterations in patients suffering from lipedema. Angiology. 1995; 46(4):333-339.
12. Forner-Cordero I, Oliván-Sasot P, Ruiz-Llorca C, Muñoz-Langa J. Lymphoscintigraphic findings in patients with lipedema. Rev Esp Med Nucl Imagen Mol. 2018; 37(6):341-348.
13. Boursier V, Pecking A, Vignes S. Comparative analysis of lymphoscintigraphy between lipedema and lower limb lymphedema. J Mal Vasc. 2004; 29(5):257-261.
14. Wold L E, Hine E A, Allen E V. Lipedema of the legs; a syndrome characterized by fat legs and edema. Ann Intern Med. 1951; 34(5): 1243.
15. Okhovat J-P, Alavi A. Lipedema. Int J Low Extrem Wounds. 2015; 14(3):262-267.
16. Shin B W, Sim Y-J, Jeong H J, Kim G C. Lipedema, a rare disease. Ann Rehabil Med. 2011; 35(6):922-927.
17. Wagner. Lymphedema and lipedema—an overview of conservative treatment. Vasa. 2011; 40(4):271-279.
18. Harvey N L, Srinivasan R S, Dillard M E, et al. Lymphatic vascular defects promoted by Prox1 haploinsufficiency cause adult-onset obesity. Nat Genet. 2005; 37(10):1072-1081.
19. Escobedo N, Oliver G. The lymphatic vasculature: Its role in adipose metabolism and obesity. Cell Metab. 2017; 26(4):598-609.
20. Petrova T V, Koh G Y. Organ-specific lymphatic vasculature: From development to pathophysiology. J Exp Med. 2018; 215(1):35-49.
21. Lin J, Li J, Huang B, et al. Exosomes: Novel biomarkers for clinical diagnosis. Sci World J. 2015; 2015:1-8.
22. Colombo M, Raposo G, Théry C. Biogenesis, secretion, and intercellular interactions of exosomes and other extracellular vesicles. Annu Rev Cell Dev Biol. 2014; 30(1):255-289.
23. Raposo G, Stoorvogel W. Extracellular vesicles: exosomes, microvesicles, and friends. J Cell Biol. 2013; 200(4):373-383.
24. van Balkom B W M, Eisele A S, Pegtel D M, Bervoets S, Verhaar M C. Quantitative and qualitative analysis of small RNAs in human endothelial cells and exosomes provides insights into localized RNA processing, degradation and sorting. J Extracell Vesicles. 2015; 4(1):26760.
25. Thakur B K, Zhang H, Becker A, et al. Double-stranded DNA in exosomes: a novel biomarker in cancer detection. Cell Res. 2014; 24(6):766-769.
26. van der Pol E, Boing A N, Harrison P, Sturk A, Nieuwland R. Classification, functions, and clinical relevance of extracellular vesicles. Mattson M P, ed. Pharmacol Rev. 2012; 64(3):676-705.
27. Valadi H, Ekström K, Bossios A, Sjostrand M, Lee J J, Lotvall J O. Exosome-mediated transfer of mRNAs and microRNAs is a novel mechanism of genetic exchange between cells. Nat Cell Biol. 2007; 9(6):654-659.
28. Taylor D D, Gercel-Taylor C. The origin, function, and diagnostic potential of RNA within extracellular vesicles present in human biological fluids. Front Genet. 2013; 4:142.
29. Kahlert C, Melo S A, Protopopov A, et al. Identification of double-stranded genomic DNA spanning all chromosomes with mutated KRAS and p53 DNA in the serum exosomes of patients with pancreatic cancer. J Biol Chem. 2014; 289(7):3869-3875.
30. Waldenström A, Gennebäck N, Hellman U, Ronquist G. Cardiomyocyte microvesicles contain DNA/RNA and convey biological messages to target cells. Qin G, ed. PLoS One. 2012; 7(4):e34653.
31. Lindstrom P. The physiology of obese-hyperglycemic mice [ob/ob Mice]. Sci World J. 2007; 7:666-685.
32. Zhang Y, Proenca R, Maffei M, Barone M, Leopold L, Friedman J M. Positional cloning of the mouse obese gene and its human homologue. Nature. 1994; 372(6505):425-432.
33. Friedman J M, Halaas J L. Leptin and the regulation of body weight in mammals. Nature. 1998; 395(6704):763-770.
34. Filipe V, Hawe A, Jiskoot W. Critical evaluation of nanoparticle tracking analysis (NTA) by NanoSight for the measurement of nanoparticles and protein aggregates. Pharm Res. 2010; 27(5):796-810.
35. Slungaard A. Platelet factor 4: A chemokine enigma. Int J Biochem Cell Biol. 2005; 37(6):1162-1167.
36. Kowalska M A, Rauova L, Poncz M. Role of the platelet chemokine platelet factor 4 (PF4) in hemostasis and thrombosis. Thromb Res. 2010; 125(4):292-296.
37. Sachais B S, Higazi A A R, Cines D B, Poncz M, Kowalska M A. Interactions of platelet factor 4 with the vessel wall. Semin Thromb Hemost. 2004; 30(3):351-358.
38. Bakogiannis C, Sachse M, Stamatelopoulos K, Stellos K. Platelet-derived chemokines in inflammation and atherosclerosis. Cytokine. 2019; 122:154157.
39. Grubbs F E. Sample criteria for testing outlying observations. Ann Math Stat. 1950; 21(1):27-58.
40. Rahier J F, Dubuquoy L, Colombel J F, et al. Decreased lymphatic vessel density is associated with postoperative endoscopic recurrence in Crohn's disease. Inflamm Bowel Dis. 2013; 19(10):2084-2090.
41. Van Kruiningen H J, Hayes A W, Colombel J F. Granulomas obstruct lymphatics in all layers of the intestine in Crohn's disease. APMIS. 2014; 122(11):1125-1129.
42. Heatley R V, Bolton P M, Hughes L E, Owen E W. Mesenteric lymphatic obstruction in Crohn's disease. Digestion. 1980; 20(5):307-313.
43. Carramolino L, Fuentes J, Garcia-Andres C, Azcoitia V, Riethmacher D, Tones M. Platelets play an essential role in separating the blood and lymphatic vasculatures during embryonic angiogenesis. Circ Res. 2010; 106(7): 1197-1201.
44. Osada M, Inoue O, Ding G, et al. Platelet activation receptor CLEC-2 regulates blood/lymphatic vessel separation by inhibiting proliferation, migration, and tube formation of lymphatic endothelial cells. J Biol Chem. 2012; 287(26):22241-22252.
45. Welsh J D, Kahn M L, Sweet D T. Lymphovenous hemostasis and the role of platelets in regulating lymphatic flow and lymphatic vessel maturation. Blood. 2016; 128(9):1169-1173.
46. Hess P R, Rawnsley D R, Jakus Z, et al. Platelets mediate lymphovenous hemostasis to maintain blood-lymphatic separation throughout life. J Clin Invest. 2014; 124(1):273-284.
47. Tabibiazar R, Cheung L, Han J, et al. Inflammatory manifestations of experimental lymphatic insufficiency. PLoS Med. 2006; 3(7):e254.
48. Maione T E, Gray G S, Petro J, et al. Inhibition of angiogenesis by recombinant human platelet factor-4 and related peptides. Science. 1990; 247(4938):77-79.
49. Tanaka T, Manome Y, Wen P, Kufe D W, Fine H A. Viral vector-mediated transduction of a modified platelet factor 4 cDNA inhibits angiogenesis and tumor growth. Nat Med. 1997; 3(4):437-442.
50. Jouan V, Canron X, Alemany M, et al. Inhibition of in vitro angiogenesis by platelet factor-4-derived peptides and mechanism of action. Blood. 1999; 94(3):984-993.
51. Lord M S, Cheng B, Farrugia B L, McCarthy S, Whitelock J M. Platelet factor 4 binds to vascular proteoglycans and controls both growth factor activities and platelet activation. J Biol Chem. 2017; 292(10):4054-4063.
52. Mueller A, Meiser A, McDonagh E M, et al. CXCL4-induced migration of activated T lymphocytes is mediated by the chemokine receptor CXCR3. J Leukoc Biol. 2008; 83(4):875-882.
53. Romagnani P, Maggi L, Mazzinghi B, et al. CXCR3-mediated opposite effects of CXCL10 and CXCL4 on TH1 or TH2 cytokine production. J Allergy Clin Immunol. 2005; 116(6): 1372-1379.
54. Zampell J C, Yan A, Elhadad S, Avraham T, Weitman E, Mehrara B J. CD4(+) cells regulate fibrosis and lymphangiogenesis in response to lymphatic fluid stasis. PLoS One. 2012; 7(11):e49940.
55. Ogata F, Fujiu K, Matsumoto S, et al. Excess lymphangiogenesis cooperatively induced by macrophages and CD4+ T cells drives the pathogenesis of lymphedema. J Invest Dermatol. 2016; 136(3):706-714.
56. Avraham T, Zampell J C, Yan A, et al. Th2 differentiation is necessary for soft tissue fibrosis and lymphatic dysfunction resulting from lymphedema. FASEB J. 2013; 27(3):1114-1126.
57. Ye L, Zhang Y-P, Yu N, Jia Y-X, Wan S-J, Wang F-Y. Serum platelet factor 4 is a reliable activity parameter in adult patients with inflammatory bowel disease: A pilot study. Medicine (Baltimore). 2017; 96(11):e6323.
58. Meuwis M-A, Fillet M, Lutteri L, et al. Proteomics for prediction and characterization of response to infliximab in Crohn's disease: a pilot study. Clin Biochem. 2008; 41(12):960-967.
59. Meuwis M-A, Fillet M, Geurts P, et al. Biomarker discovery for inflammatory bowel disease, using proteomic serum profiling. Biochem Pharmacol. 2007; 73(9):1422-1433.
60. Simi M, Leardi S, Tebano M T, Castelli M, Costantini F M, Speranza V. Raised plasma concentrations of platelet factor 4 (PF4) in Crohn's disease. Gut. 1987; 28(3):336-338.
61. Yoshida H, Yilmaz C E, Granger D N. Role of tumor necrosis factor-α in the extraintestinal thrombosis associated with colonic inflammation. Inflamm Bowel Dis. 2011; 17(11):2217-2223.
62. Danese S, De La Motte C, Fiocchi C. Platelets in inflammatory bowel disease: Clinical, pathogenic, and therapeutic implications. Am J Gastroenterol. 2004; 99(5):938-945.
63. Harries A D, Fitzsimons E, Fifield R, Dew M J, Rhoades J. Platelet count: A simple measure of activity in Crohn's disease. Br Med J. 1983; 286(6376):1476.
64. Kovi J, Duong H D, Hoang C T. Ultrastructure of intestinal lymphatics in Crohn's disease. Am J Clin Pathol. 1981; 76(4):385-394.
65. Wang X, Zhao Z, Zhu K, et al. Effects of CXCL4/CXCR3 on the lipopolysaccharide-induced injury in human umbilical vein endothelial cells. J Cell Physiol. 2019; 234(12):22378-22385.
66. Tian W, Rockson S G, Jiang X, et al. Leukotriene B₄ antagonism ameliorates experimental lymphedema. Sci Transl Med. 2017; 9(389):eaa13920.
67. Di Gennaro A, Kenne E, Wan M, Soehnlein O, Lindbom L, Haeggström J Z. Leukotriene B4-induced changes in vascular permeability are mediated by neutrophil release of heparin-binding protein (HBP/CAP37/azurocidin). FASEB J. 2009; 23 (6): 1750-1757.
68. Escobedo N, Proulx S T, Karaman S, et al. Restoration of lymphatic function rescues obesity in Prox1-haploinsufficient mice. JCI insight. 2016; 1(2):1-30.
69. Ewart-Toland A, Mounzih K, Qiu J, Chehab F F. Effect of the genetic background on the reproduction of leptin-deficient obese mice. Endocrinology. 1999; 140(2):732-738.
70. Wigle J T, Oliver G. Prox1 function is required for the development of the murine lymphatic system. Cell. 1999; 98(6):769-778.
71. Child A H, Gordon K D, Sharpe P, et al. Lipedema: An inherited condition. Am J Med Genet Part A. 2010; 152(4):970-976.
72. Wieczorek S, Combes F, Lazar C, et al. DAPAR & ProStaR: software to perform statistical analyses in quantitative discovery proteomics. Bioinformatics. 2017; 33 (1): 135-136.
73. Bϕ T H, Dysvik B, Jonassen I. LSimpute: accurate estimation of missing values in microarray data with least squares methods. Nucleic Acids Res. 2004; 32(3):e34.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method of treatment, comprising:
identifying a subject as having a disease or condition of the lymphatic vasculature based on an increased expression of platelet factor 4 (PF4) in a sample from the subject; and
administering to the subject a treatment for the disease or condition of the lymphatic vasculature.

2. The method of claim 1, wherein the sample comprises a bodily fluid.

3. The method of claim 1, wherein the sample comprises exosomes from blood plasma.

4. The method of claim 1, wherein the sample comprises blood plasma.

5. The method of claim 1, wherein the sample comprises exosomes from blood plasma.

6. The method of claim 1, wherein step (a) comprises measuring the expression of PF4 protein.

7. The method of claim 1, wherein step (a) comprises measuring the expression of PF4 protein.

8. The method of claim 1, wherein the disease or condition of the lymphatic vasculature is lymphedema, lipedema, or lymphovascular disease.

9. The method of claim 1, wherein the subject has previously been diagnosed with obesity.

10. The method of claim 1, wherein the patient has one or more of the following: swelling of part of an arm, leg, breast, or torso; a feeling of heaviness or tightness in part of an arm, leg, breast, or torso; restricted range of motion in an arm or leg; fibrosis; numbness or tingling in an arm or leg; infection; disproportionate fat below the waist.

11. The method of claim 1, wherein the subject has suffered lymphatic trauma, or has undergone a surgery, or has undergone a cancer treatment.

12. The method of claim 13, wherein the cancer treatment comprises one or more of: lymph node removal and radiation therapy.

13. The method of claim 1, wherein the subject has been diagnosed with a disease or condition of the lymphatic vasculature selected from the group consisting of obesity, inflammatory bowel disease/Crohn's disease, cellulitis, cancer, glaucoma, some forms of neural pathology, Hodgkin's Disease/Hodgkin's Lymphoma, Non-Hodgkin's Lymphoma, lymphadenitis, lymphangitis, lymphocytosis, lymphatic vascular malformation, chylous effusions, central conducting lymphatic anomaly, Kaposiform lymphangiomatosis, protein-losing enteropathy.

14. The method of claim 1, wherein the treating comprises one or more of exercise, wrapping the subject's affected limb with bandages to encourage lymph fluid to flow back toward the trunk; massage to promote lymph drainage; pneumatic compression to promote lymph drainage; wearing compression garments; and complete decongestive therapy (CDT).

15. The method of claim 1, wherein the treating comprises administration of an agent blocks PF4 signaling.


SEQUENCE LISTING (4 sequences)

SEQ ID NO: 1 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 2 - protein, 110 aa, Homo sapiens
MITATLNGEPAECLATVPGAAPAPPTWLEQLLSGGGVIYAEAEEDGDLQCLCVKTTSQVRPRHITSLEVIKAGPHCPTAQLIATLKNGRKICLDLQAPLYKKIIKKLLES

SEQ ID NO: 3 - DNA, 773 bp, Homo sapiens
attggccacagagacccagcccgagtttcccatcgcactgagcactgagatcctgctggaagctctgccgcagcatgagctccgcagccgggttctgcgcctcacgccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaacttctgatatttgtgttatccttatgattttaaataaacaaaataaatcaagttgtagtatagtcaaaatacttcttaataatagtgcaaaaattgtgttgacacataacaatttcatggaagaaaaaaattccggtattttaagcaaaaagtattttgaaggaaggtgtgaatactggttatgcttggtgttacatgttggctgatacatattcatgcatttacatgattgcagtactttatagctacatatttaccttgaccattattattacctttgccaataaatatcagtaacacagatggcttttaaaaaa

SEQ ID NO: 4 - DNA, 1061 bp, Homo sapiens
atcttagtttccgcaccgcagttcctcggtgtccacttcaggcttccggactggaaggacagccgggaataaaacgtgccggcgaggctcaggagtcattggccacagagacccagcccgagtttcccatcgcactgagcactgagatcctgctggaagctctgccgcagcatgagctccgcagccgggttctgcgcctcacgccccgggctgctgttcctggggttgctgctcctgccacttgtggtcgccttcgccagcggtgagagcagaagccaggctgtgagggctggcagcggcgagggggagtccgggaagccctggggctggggaggaatcctctaggatcatgatcacagccacacttaacggagagcctgctgagtgtctggccacagtgccaggcgctgcacctgcacctcccacctggttagaacaacttctgtctgggggaggtgtgatttatgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaacttctgatatttgtgttatccttatgattttaaataaacaaaataaatcaagttgtagtatagtcaaaatacttcttaataatagtgcaaaaattgtgttgacacataacaatttcatggaagaaaaaaattccggtattttaagcaaaaagtattttgaaggaaggtgtgaatactggttatgcttggtgttacatgttggctgatacatattcatgcatttacatgattgcagtactttatagctacatatttaccttgaccattattattacctttgccaataaatatcagtaacacaga